(19) United States
(12) Patent Application Publication
(10) Pub. No.: US 2011/0182946 A1
(43) Pub. Date: Jul. 28, 2011

(54) Formation of Nanostructured Particles of Poorly Water Soluble Drugs and Recovery by Mechanical Techniques

(75) Inventors: Keith P. Johnston, Austin, TX (US); Robert O. Williams, III, Austin, TX (US); Michal E. Matteucci, Austin, TX (US)

(73) Assignee: BOARD OF REGENTS, THE UNIVERSITY OF TEXAS SYSTEM, Austin, TX (US)

(21) Appl. No.: 12/933,405

(22) PCT Filed: Mar. 17, 2009
(86) PCT No.: PCT/US09/37391

§ 371 Date: Apr. 5, 2011

Related U.S. Application Data
(60) Provisional application No. 61/037,213, filed on Mar. 17, 2008.

(51) Int. Cl. A61K 9/14; A61K 31/496; A61K 31/192; A61P 29/00; A61P 31/00; B82Y 5/00

(52) U.S. Cl. 424/400; 514/254.07; 514/570; 977/773

(57) ABSTRACT

The present invention provides a composition and method of forming an amorphous drug-loaded particle by forming one or more amorphous drug-loaded nanoparticles comprising one or more active agents stabilized by one or more polymers, desolvating the one or more amorphous drug-loaded nanoparticles to form one or more flocculated amorphous drug-loaded nanoparticles, filtering the one or more flocculated amorphous drug-loaded nanoparticles and drying the one or more flocculated amorphous drug-loaded nanoparticles to form amorphous drug-loaded particles.

### CROSS-REFERENCE TO RELATED APPLICATIONS

This application is a National Stage Application of International Application No. PCT/US2009/037391, filed Mar. 17, 2009, which claims priority to U.S. Provisional Application Ser. No. 61/037,213, filed Mar. 17, 2008, the contents of each of which are incorporated by reference herein in their entireties.

### TECHNICAL FIELD OF THE INVENTION

The present invention relates in general to the field of particle formation, and more specifically to the formation of nanostructured particles of poorly water soluble drugs and recovery by mechanical techniques.

### BACKGROUND ART

Without limiting the scope of the invention, its background is described in connection with nanostructure particle formation, more particularly, to a process consisting of rapidly flocculating polymerically stabilized nanoparticles.

It is estimated that more than one-third of the compounds being developed by the pharmaceutical industry are poorly water-soluble. The bioavailability of class II poorly water-soluble drugs is often dependent upon the dissolution rate of the drug in the gastrointestinal tract. Dissolution rates may be increased by reducing the particle size to increase the surface area, and by coating drug particles with hydrophilic surfactants to enhance wetting and solvation.

Antisolvent precipitation is a widely used process to prepare inorganic and organic particles including nanoparticles of poorly water-soluble drugs. In this process, a poorly water-soluble drug with or without surfactant(s) is dissolved in a water miscible organic solvent, such as methanol, ethanol, tetrahydrofuran (THF) and acetonitrile etc. The organic solution is then mixed with an “antisolvent”, usually an aqueous solution containing a surfactant(s) by a confined impinging jets (CIJ) mixer, controlled precipitation, sonication, or direct addition (pouring) of the antisolvent into organic drug solution. Supercritical fluid antisolvents can be used to achieve a great deal of control over particle morphology, as a result of rapid two-way diffusion. Upon mixing, the supersaturation and concentration of stabilizing surfactants may be controlled to manipulate the nucleation and growth of drug particles. With sufficient supersaturation, and arrested growth by surfactant stabilization, it becomes possible to form suspensions of submicron particles in the aqueous solution.

Several drying techniques have been used to recover drug nanoparticles from aqueous suspensions produced by antisolvent precipitation and other methods. Ketoconazole, itraconazole and ibuprofen micronized particles were spray dried. Budesonide particles with a size range from 1 to 10 μm were filtered with 0.8 μm-pore-size polycarbonate membrane. In addition, a continuous, evaporative recovery method was used to strip the organic solvent, and then the aqueous suspension was spray dried.

For a large fraction of newly discovered drugs, bioavailability is limited by poor solubility in water. Amorphous nanoparticles with high surface area may be designed to raise dissolution rates as well as to achieve high levels of supersaturation. An increase in the supersaturation in the gastrointestinal tract would lead to greater flux through biomembranes and higher bioavailability. Solubilities of amorphous drugs may reach 1600-times that of the crystalline form. Typically, amorphous solid solutions or dispersions of drugs, stabilized by high glass transition (T_{g}) polymers, are formulated by co-grinding, solvent evaporation, or hot melt extrusion. Amorphous formulations, however, have a tendency to crystallize during dissolution. This crystallization may be minimized by designing rapidly dissolving nanoparticles, with surface areas on the order of 50 m²/g, particularly for slowly dissolving stabilizers such as hydroxypropylmethylcellulose. High surface area amorphous micro- and nanoparticles may be formed by rapid nucleation from solution along with arrested growth. For example, in antisolvent precipitation of organic solutions mixed with aqueous media, preferential adsorption of the stabilizer at the particle surface inhibits nanoparticle growth and crystallization even with drug loadings of 94% (drug wt./tot. wt.).

A wide variety of techniques have been developed to form inorganic and pharmaceutical nanocrystals in the presence of stabilizers. Techniques include mechanical milling, changing physical solvent properties, for example, antisolvent precipitation, and by chemical reactions, such as thermal decomposition. However, the recovery of the nanoparticles from solution and further processing of the nanocrystals in the solid state remains a formidable challenge. Common techniques for solvent removal include spray drying, freeze drying and ultrafiltration. Spray drying is energy intensive and yields can be limited. The elevated temperatures of 90° C. or higher, and an increase in volume fraction of the particles during droplet shrinkage, may cause crystallization or particle growth by the increase in the collision rates and/or Ostwald ripening. In addition, loss in solvent quality at elevated temperatures may lead to collapse of the polymeric steric stabilizers. Tray lyophilization requires extremely long processing times. During freezing processes, particles are concentrated in the unfrozen aqueous phase as ice crystals are removed. Again the increase in particle concentration and changes in solvent quality may cause particle crystallization and growth. Ultrafiltration of nanoparticles has also been used to purify nanoparticle dispersions; however, filtrate removal rates were limited to approximately 0.03 mL/min·cm², even for a membrane surface area of 1000 cm², and the dispersion could only be concentrated by a factor of ⅕. Long filtration times allow for particle growth via Ostwald ripening and coagulation. It would be desirable to develop novel rapid techniques for particle recovery and solvent removal to avoid undesirable crystallization and growth and to increase production rates of dry powders in a useful morphology for subsequent processing.

An alternative approach is to flocculate nanoparticles reversibly to form large flocs that may be filtered rapidly. Flocculation of negatively charged gold nanoparticles with positively charged poly(L-lysine) created spherical sub-micron aggregates, which formed unique 1-5 μm hollow spheres upon subsequent addition of SiO₂ nanoparticles. Flocculation may also be produced by adding electrolytes to weaken the hydration of the stabilizers on the nanocrystal surface, resulting in interparticle attractive interactions. Electrolytes decrease the lower critical solution temperature (LCST) of poly(ethyleneoxide) (PEO) chains by inducing desolvation of the ether oxygens. The LCST corresponds to the critical flocculation temperature (CFT) of the particle dispersion for micron sized particles stabilized by PEO containing copolymers. Recently, researchers utilized Na₂SO₄ to flocculate crystalline naproxen nanoparticles stabilized by poly(vinylpyrrolidone) or PEO chains. Aqueous suspensions (50 ml) of the large flocs were filtered in minutes to obtain a dry powder. The dried powders were redispersible to the, original 300 nm particle size and drug yields after filtrations were as high as 99% (wt. recovered drug/wt. input drug). Alternatively, addition of Na₂SO₄ or KCl to sterically stabilized platinum catalyst nanoparticles induced instability as evident by turbidimetry. However, these nanoparticles were not filtered or shown to redisperse to the original size.

Thus, the present inventors recognized that there is a need in the field for a new method to produce flocs of amorphous polymer-stabilized nanoparticles. The present invention addressed the need.

### DISCLOSURE OF THE INVENTION

The present invention related to the formation of amorphous nanoparticle aggregates by a flocculation and filtration process. These aggregates can give improved properties for forming and maintaining supersaturated solutions that can enhance bioavailability of these drugs. Even though the salt flocculation process is fairly well known and reported by Chen et al., the desirable properties of the harvested particles were not anticipated.

The previous work by Chen has shown that salt flocculation of crystalline nanoparticle dispersions creates dried crystalline particles that redisperse to their original size (about 300 nm diameter), according to SEM and static light scattering measurements. In certain embodiments, the current invention is the processing of amorphous rather than crystalline nanoparticles from aqueous dispersions while maintaining the amorphous morphology. Furthermore, the new salt flocculation process can reduce the surface area of polymerically stabilized amorphous nanoparticles as the particle size increased by about an order of magnitude. In contrast, the particle size remained constant in the work of Chen.

In a certain embodiment, the present invention demonstrates the supersaturated conditions are obtainable for a poorly-water soluble compound in an aqueous media. These conditions are maintained over an extended period of time. The crystalline particles as described by Chen et al. do not form supersaturated solutions. Supersaturated solutions are known to improve bioavailability of poorly water-soluble drugs.

In an embodiment, the process consists of rapidly flocculating polymerically stabilized nanoparticles by the addition of salt to or change in pH of the dispersion medium. The flocculated amorphous nanoparticle dispersion can then be rapidly filtered to remove water, additional solvents, excess unbound stabilizers and soluble salts. Rinsing the filter cake with a polymer aqueous solution minimizes the residual salt remaining to less than 1% of the total weight of the dried final particles. Rinsing with a polymeric aqueous solution was not described by Chen et al.

The current invention shows the application of salt or pH flocculation to polymerically stabilized amorphous nanoparticles as a means to control the reduction in particle surface area by irreversible aggregation. STEM is used to illustrate that the aggregates formed by salt or pH flocculation contain primary particle sizes below 1 μm, however their size upon redispersion is 2-10 μm according to static light scattering and corroborated by SEM and BET. The concept of controlled particle growth resulting from irreversible aggregation by specific selection of stabilizers and type of flocculation is not taught by Chen et al.

The preservation of the amorphous morphology throughout the salt and pH flocculation process is not obvious, since typical filtration of nanoparticles takes a period of time sufficient for crystallization. Crystallization of amorphous particles is accelerated by the presence of water and other solvents. However, salt and pH flocculation produce nanoparticle aggregates which separate from the bulk aqueous phase by creaming or settling into a second layer. This separated bulk phase aids in the isolation of particles from water and solvent, which minimizes the time the particles are susceptible to crystallization. Additionally, the flocculation and filtration is conducted at temperatures well below the glass transition temperature of the poorly water-soluble compound (i.e. itraconazole, 58° C.) to minimize mobility of the drug molecules and thus preserve the amorphous morphology even during the additional washing step. In addition, the ability to use rapid filtration and drying eliminates the need to lyophilize the aqueous dispersion to produce a dry powder. Avoiding lyophilization increases the variety of solvents and soluble salts that can be used to initially produce the metastable amorphous nanoparticles. The preservation of amorphous morphology throughout the filtration and additional washing steps was not obvious or anticipated in the work of Chen at al., since only crystalline particles were discussed in the work.

The present invention provides drug particles that are flocculated and filtered to recover amorphous nanoparticles. The previous technology teaches formation of crystalline nanoparticles. Amorphous particles give higher supersaturation needed for higher bioavailability. The flocculation may be controlled. In one embodiment, after redissolution of the flocs, the particle size can be larger than the original size; in contrast, the work of Chen et al. and another embodiment of this invention teaches the particle size will be the original size. The larger size has advantages in particle processing and in producing higher supersaturation as shown in the examples. In addition, the present invention provides for rinsing for removal of the salt without crystallizing the particles. The present invention maintains the particle size and even allows control of the particle size; in contrast, other harvesting techniques such as spray drying and freezing drying don't allow control over the particle size.

In some embodiments, the current invention also claims that controlling the growth of particle size, to reduce the surface area of the polymerically stabilized nanoparticles, helping to improve and maintain the level of supersaturation attained upon dissolution. Rapidly dissolving amorphous nanoparticles have the potential to raise supersaturation values markedly, relative to more conventional low surface area (<1 m²/g) solid dispersions, by avoiding solvent-mediated crystallization of the undissolved solid drug domains. An unanticipated and non-obvious result is that the nanoparticle aggregates created by salt and pH flocculation dissolve as rapidly as individual nanoparticles of the same composition. Additionally, the nanoparticle aggregates produced by salt or pH flocculation are particularly effective for maintaining high supersaturations for several hours, compared to individual nanoparticles, by decreasing the number of heterogeneous sites for nucleation and growth of particles out of the solution. Therefore, the irreversible aggregation of nanoparticles improves the ability of the amorphous drug to supersaturate and maintain levels of supersaturation in aqueous media. Previous work by Chen et al. showed crystalline particles that will not supersaturate aqueous media. However, theoretically if Chen's particles were amorphous, they would still redisperse to their original size and therefore no change in the supersaturation curve would be observed relative to the original nanoparticle dispersion. Thus, the advancement in dissolution of amorphous nanoparticles is not taught by the previous work of Chen et al. In some embodiments, the polymeric stabilizers may include both non-ionic and pH dependent release polymers flocculated by desolvating the stabilizing moieties by either the addition of a divalent salt or shifting the pH. The use of pH flocculation offers a new type of controlled release through the use of enterically coated amorphous nanoparticle aggregates. Therefore, the release of supersaturation may be tuned by control of the aggregate surface area and the choice of enteric polymer. The use of pH dependent release polymers was not discussed in the previous work by Chen et al. and is a second reduction to practice of the current invention.

Overall, the present invention produces a method to improve the ability of poorly water-soluble particles to supersaturate aqueous media. The controlled and irreversible aggregation that lead to growth of the nanoparticles, the ability to maintain amorphous morphology even through the additional washing step, the increase in overall ability to supersaturate aqueous media for prolonged periods of time, and a second reduction to practice with flocculation by changing the pH were all unanticipated results.

The present invention provides a method of forming an amorphous drug-loaded particle by forming one or more amorphous drug-loaded nanoparticles, desolvating the one or more amorphous drug-loaded nanoparticles to form one or more flocculated amorphous drug-loaded nanoparticles, filtering and drying the one or more flocculated amorphous drug-loaded nanoparticles to form amorphous drug-loaded particles. The one or more amorphous drug-loaded nanoparticles include one or more active agents stabilized by one or more polymers.

The present invention also provides a flocculated drug-loaded amorphous nanoparticle. The flocculated drug-loaded amorphous nanoparticle includes one or more active agents and one or more polymer stabilizers that have been desolvated to form one or more flocculated amorphous drug-loaded nanoparticles that form a supersaturated solution of one or more drug-loaded amorphous nanoparticles when resuspended.

A method of increasing the bioavailability of an active agent in a subject is provided by the present invention. The method includes administering to a subject an amorphous drug-loaded floc having one or more active agents and one or more polymer stabilizers that have been desolvated to form one or more flocculated amorphous drug-loaded nanoparticles that form a supersaturated solution of one or more drug-loaded amorphous nanoparticles when resuspended.

Similarly, the present invention includes a method of increasing the concentration of an active agent in a subject administering to a subject one or more flocculated amorphous drug-loaded particles comprising one or more active agents and one or more polymer stabilizers that have been desolvated to form one or more flocculated amorphous drug-loaded nanoparticles that form a supersaturated solution of one or more drug-loaded amorphous nanoparticles when administered to a subject.

The present invention includes an amorphous drug-loaded particle floc formed by the process of forming one or more amorphous drug-loaded nanoparticles, desolvating the one or more amorphous drug-loaded nanoparticles to form one or more flocculated amorphous drug-loaded nanoparticles. The one or more flocculated amorphous drug-loaded nanoparticles are then filtered and dried to form amorphous drug-loaded particles. The one or more flocculated amorphous drug-loaded nanoparticles achieve a supersaturated solution when resuspended. The one or more amorphous drug-loaded nanoparticles include one or more active agents stabilized by one or more polymers.

Furthermore, the present invention includes a method of forming a redispersible floc by forming one or more amorphous drug-loaded nanoparticles with one or more active agents stabilized by one or more polymers, desolvating the one or more amorphous drug-loaded nanoparticles to form one or more flocculated amorphous drug-loaded nanoparticles, filtering the one or more flocculated amorphous drug-loaded nanoparticles and drying the one or more flocculated amorphous drug-loaded nanoparticles to form amorphous drug-loaded particles.

Dry powder obtained from aqueous dispersions of nanoparticle aggregates, formed by antisolvent precipitation, dissolved to form highly supersaturated solutions in 0.17% sodium dodecyl sulfate solution (pH 6.8). Medium surface area 2-5 m²/g particles were produced by salt flocculation/filtration of ITZ/hydroxypropylmethylcellulose (HPMC) dispersions, whereas high surface area 13-36 m²/g particles were produced by controlled precipitation followed by lyophilization. Both types of particles dissolved rapidly to produce supersaturation levels up to 17 in 10 minutes. However, the decay in supersaturation from the maximum value over four hours was much slower for the medium surface area particles, as the smaller excess surface area of undissolved particles led to slower nucleation and growth from solution. A similar result was achieved by initially dissolving part of the drug at pH 1.2 to reduce the excess surface area of undissolved particles, and then shifting the pH to 6.8. This pH shift mimics the transition from the stomach to the intestines. The ability to control the particle morphology and supersaturations generated and sustained at pH 6.8 offers new opportunities in raising bioavailability in gastrointestinal delivery.

### DESCRIPTION OF THE DRAWINGS

For a more complete understanding of the features and advantages of the present invention, reference is now made to the detailed description of the invention along with the accompanying FIGURES and in which:

FIG. 1 is a schematic diagram showing the process for producing pharmaceutical powder by antisolvent precipitation, flocculation with salt, filtration and vacuum drying;

FIG. 2 is a plot of the temperature effect on particle size of naproxen suspensions produced by antisolvent precipitation;

FIG. 3 is a plot of the cloud point temperature of PVP and F127 at various sodium sulfate concentrations in water;

FIG. 4(a) is a plot of the effects of salt concentration on dissolution rate of flocculated, filtered and vacuum dried naproxen nanoparticles produced by antisolvent precipitation (system B);

FIG. 4(b) is a plot of the effect of stabilizers on dissolution rate of flocculated, filtered and vacuum dried naproxen nanoparticles produced by antisolvent precipitation;

FIG. 4(c) is a plot of the correlation between dissolution rate and specific surface area of flocculated, filtered and vacuum dried naproxen particles (R²=0.97);

FIG. 5(a) is a microscopic image of naproxen flocs of system B in suspension at salt concentration of 1.01M;

FIG. 5(b) are SEM images of naproxen flocs after fillration and vacuum drying.

FIG. 6 is a picture of X-ray diffraction of flocculated, filtered and vacuum dried naproxen particles produced by antisolvent precipitation;

FIGS. 7(A)-7(D) are pictures of progression of salt flocculation. FIG. 7(A) depicts original dispersion, FIG. 7(B) depicts 3 sec. after salt solution addition, FIG. 7(C) depicts 3 minutes after salt solution addition, and FIG. 7(D) depicts after dry powder redispersion in pure water at about 10 mg/mL;

FIG. 8 is an image taken using optical microscopy of 8:1:2 ITZ/P407/HPMC dispersion just after addition of salt solution;

FIG. 9 is a reversible heat flow diagram from modulated differential scanning calorimetry: HPMC-stabilized, salt flocculated powders;

FIG. 10 is a heat flow diagram from the modulated differential scanning calorimetry: HPMC-stabilized, salt flocculated powder;

FIG. 11 is a heat flow diagram from the modulated differential scanning calorimetry: HPMC/P407-stabilized, salt flocculated powders and spray dried, homogenized, and rapidly frozen controls;

FIG. 12 is a plot of supersaturation versus time of salt flocculated powders in pH 6.8 media with 0.17% SDS, compared with lyophilized and original dispersion before flocculation;

FIG. 13 is a plot of floc structure as a function of polymer solvation and (I);

FIG. 14 is a SEM image of salt flocculated ITZ dispersions after rapid freezing onto aluminum stage;

FIG. 15 is a plot of supersaturation of basic media from salt flocculated itraconazole nanoparticles;

FIG. 16 is a plot of the heat flow versus the temperature detailing the Morphology of Salt Flocculated Itraconazole Nanoparticles;

FIG. 17 is an illustration of the flocculation process; and

FIG. 18 is a plot of the average plasma concentration of ITZ in rats.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention relates to a new process for rapidly separating nanoparticles of chemicals (pharmaceuticals, agricultural chemicals, nutraceuticals) from an aqueous suspension or dispersion where the dried particles may then be redispersed in water or other polar solvents to their original size and morphological form (for example, amorphous or a specific crystalline structure). The particle recovery process involves agglomerating the nanoparticles to a larger flocculate, in order to be rapidly separated by standard methods, such as filtration or centrifugation. Flocculation may be induced by adding a flocculant to the aqueous suspension. It may also be induced by changing the temperature or chemical composition of the solution, for example by changing pH or ionic strength. The aggregates may then be filtered, centrifuged or separated by other mechanical means and formulated into a dosage form. The particles may then be recovered and redispersed into an aqueous environment where the mean particle size is the same, or 10%, or 20%, or 50% or 100% or 500% or 1000% larger. After separation, the particles are able to achieve enhanced solubility equal to or better than an identical formulation isolated by conventional means, such as freeze drying or spray drying. The flocculated particles can exhibit slower release kinetics than the original nanoparticles, allowing controlled release of the chemical agent. In addition the particles may be mixed with other excipients to make pharmaceutical dosage forms including tablets, gels and capsules.

Previous processes used spray drying, evaporation, freeze drying, or spray freezing to isolate nanoparticles from suspension. These processes can have a detrimental impact on the morphology of the particles, depending on the Tg of the particles relative to the processing temperature. For example the addition of heat or solvent removal may cause growth in the primary particle size or crystallization of amorphous domains. The present invention is a process to control the aggregation of the nanoparticles with excipients and stabilizers. Furthermore, the aggregates may be formed at temperatures below ambient and the filtration may also be performed at low temperature to prevent particle growth or changes in the polymorphism. The aggregates may then simply be filtered or centrifuged or mechanically separated from the solution. It is now no longer necessary to evaporate or freeze the solvent. The aggregation may be accomplished by adding surface active chemical agents, polymers, binders, flocculants or gelling agents. In some cases the solution may be cooled to temperatures as low as 0° C., or even −50° C. to aid this aggregation. The aggregation is controlled in such a manner to produce a redispersible powder with the same morphological as the original particles in suspension. Additionally, the excess excipient, which is free in the filtrate, is removed from the particles prior to drying, which increases the drug loading of the final product. This aspect of the invention allows large amount of excipients to be used during the particle formation step, which are then removed from the final form where they are no longer needed.

For oral therapeutic administration, the particles containing the active compound(s) may be incorporated with excipients and used in the form of ingestible tablets, buccal tablets, troches, capsules, elixirs, suspensions, syrups, wafers. Such compositions and preparations should contain at least 0.1 percent of active compound. The percentage of the compositions and preparations may, of course, be varied and may conveniently be between 2 to 60 percent of the weight of the unit. The amount of particles containing the active compound(s) in such therapeutically useful compositions is such that a suitable dosage will be obtained.

One aspect of this present invention's separation process is that it may be utilized without changing the morphology of the drug particles. For example, amorphous nanostructured particles may be maintained throughout the precipitation and separation process. Previous work by Chen et al. has shown flocculation process with crystalline particles only, where recovery was greater than 90% and redispersion sizes were nearly identical to the original suspension. The ability of amorphous or higher energy crystalline polymorphs to supersaturate aqueous media, relative to their most stable crystalline counterparts, is maintained after particle isolation. Comparison to identical formulations isolated by lyophilizations shows better supersaturation levels from particles separated by the current invention. Other isolation techniques, such as spray drying, must be conducted at elevated temperatures, which could lead transition of an amorphous form or less stable crystalline polymorph to the most stable crystalline form. Filtration with submicron sized membranes and freeze drying are lengthy procedures, which allow ample time for nanoparticles to grow via Ostwald ripening and solvent-mediated crystallization, leading to slower dissolution rates and lower solubility.

In certain embodiments, the present invention's process enables the enhancement of solubility and rate of dissolution of a chemical agent and provides an alternative to other routinely employed techniques to enhance solubility and rate of dissolution, such as mechanical pulverization, airjet micronization, ball milling, amorphous glass process, solid solution process, and solid dispersion process. It should be recognized that the techniques mentioned previously have limited application to enhancement of solubility and rate of dissolution for water insoluble chemical agents. For instance, the use of mechanical pulverization typically achieves a size range of greater than 50 microns and a polydisperse particle size distribution. The current invention is capable of producing much smaller particle sizes and more monodisperse particle size distributions as well as polymorphs which enhance aqueous solubility. In addition, some of the techniques mentioned previously expose the chemical agent (active drug substance) to conditions (high temperature, mechanical shear) that may cause chemical degradation, rendering the final product (active drug substance) inactive.

This process can be used to produce nanoparticles and microparticles containing pharmaceutical drug substances that are insoluble or poorly soluble in water. This will enhance the solubility and rate of dissolution of the drug substance, and enhance the bioavailability. In the pharmaceutical industry, most new chemical entities being developed are poorly water-soluble. Although there are several routinely used processes in the pharmaceutical industry to reduce the particle size, each has their limitations (see previous discussion). In addition, this process can be used to produce agricultural chemicals. The chemical agents are typically poorly water-soluble and therefore must be formulated in much higher concentrations for adequate activity. This process may enable incorporation of these agents at lower concentrations in the agricultural chemical product so that lower amounts would be required to produce the same effect. The aggregation must be controlled. This can be done with stabilizers and temperature, pH and ionic strength.

One model drug of choice that may be used as example is Naproxen, d-2-(6-methoxy-2-naphthyl)propionic acid, CH₃OC₁₀H₆CH(CH₃)CO₂H, (as free acid) is an anti-inflammatory compound with a low water solubility of 15 mg/L.

One objective of this invention is to recover nanoparticles of poorly water-soluble drugs produced by antisolvent precipitation at low temperatures from 0 to 22° C. by flocculation and filtration, and to examine their properties. Temperature is shown to have a large effect on the particle size distribution in the suspension. The particles were separated from the solvent by flocculation with a concentrated salt solution, to aggregate the steric stabilizers. The flocculated particles were recovered from the aqueous solution by filtration and were then vacuum dried. The surfactant composition and structure, and the type and concentration of salt in the drug suspension were optimized to produce large, loose flocs, which could be redispersed into pure water readily after filtration and drying. The increase in drug potency upon filtration and the reproducibility in the composition and yield of the powder were examined. Another goal of the present invention is to achieve particle sizes after redispersion, which was similar to those in the original suspension prior to flocculation. The dissolution rate of naproxen powder produced by this technique was compared with that of identical aqueous drug suspensions dried by lyophilization. X-ray diffraction was used to investigate the crystallinity of naproxen. Optical microscopy and SEM were used to characterize the morphologies of the naproxen flocs in the suspension and after drying. The concentration of residual salt in the dried samples was measured by conductivity and shown to be far below the toxic limit. Compared to other solvent removal techniques, the use of flocculation and filtration offers several advantages. The flocs may be filtered much more rapidly and efficiently than the original nanoparticle suspension. Rapid filtration reduces the time for the growth of the primary particles in the concentrated precipitate. Particle growth is a potential problem in this step as the stabilizers on the particle become less solvated as the filtrate is removed. The filtration can be operated at low temperatures more easily than in the case of spray drying and other solvent evaporation techniques. For example, drying at high temperatures may lead to undesirable particle growth. Separation by filtration avoids challenges in evaporation, for example for solvents such as ethanol that form azeotropes with water. Whereas it may take days for lyophilization, the time for flocculation and filtration will be shown to be on the order of minutes. Finally, the potency of the drug can be increased in the filtration step since the dispersed particle phase contains a higher fraction of drug than the continuous phase, as soluble surfactant stabilizers are removed with the filtrate.

Another objective of the present invention is to produce flocs of amorphous polymer-stabilized nanoparticles, which may be filtered, dried, and redispersed, to achieve (1) the original primary particle sizes, and (2) rapid generation of supersaturated solutions up to 14-times the crystalline solubility, despite drug loadings up to 94%. The present invention expands the flocculation of nanoparticle crystalline drugs to include amorphous morphologies and higher energy crystalline states. Comparing the mechanisms of particle aggregation and redispersion for salt flocculation relative to spray drying and rapid freezing as a function of changes in particle volume fraction φ and solvent quality for the stabilizers shows that while φ increases in spray drying and rapid freezing during water removal, it remains constant during salt flocculation. The differences in the pathways in particle volume fraction and solvent conditions, as a function of temperature and salinity, will be shown to have a profound effect on the particle size upon redispersion and level of supersaturation as the particles dissolve. The rapid change of interparticle forces from repulsive to attractive, upon addition of salt, produces open flocs with low fractal dimensions. Consequently, the flocs redisperse rapidly to the original primary particle size in water. The size of the redispersed particles will be shown to be much smaller than for the more compact flocs produced by freezing/lyophilization. Additionally, low temperatures and rapid removal of solvent during filtration will be utilized to preserve the amorphous morphology of the nanoparticles more effectively than in the case of spray drying, as demonstrated by modulated differential scanning calorimetry and by high supersaturation levels in pH 6.8 media. The rapid dissolution of nanoparticles recovered by salt flocculation prevents significant crystallization of the undissolved solid phase during dissolution, leading to high supersaturation values, with the potential to enhance bioavailability.

Naproxen, ketoconazole, polyvinylpyrrolidone (PVP K-15, MW=10,000 Da) and poly(ethylene oxide-b-propylene oxide-b-ethylene oxide) (poloxamer 407) with a nominal molecular weight of 12,500 Da and a PEO/RPO ratio of 2:1 by weight were purchased from Spectrum Chemical Mfg. Corp. Sodium sulfate (anhydrous) and sodium carbonate (anhydrous) were obtained from Fisher Scientific Company. Sodium phosphate tribasic dodecahydrate was from EM Science. HPLC grade acetonitrile and methanol were obtained from EM Industrial Inc.

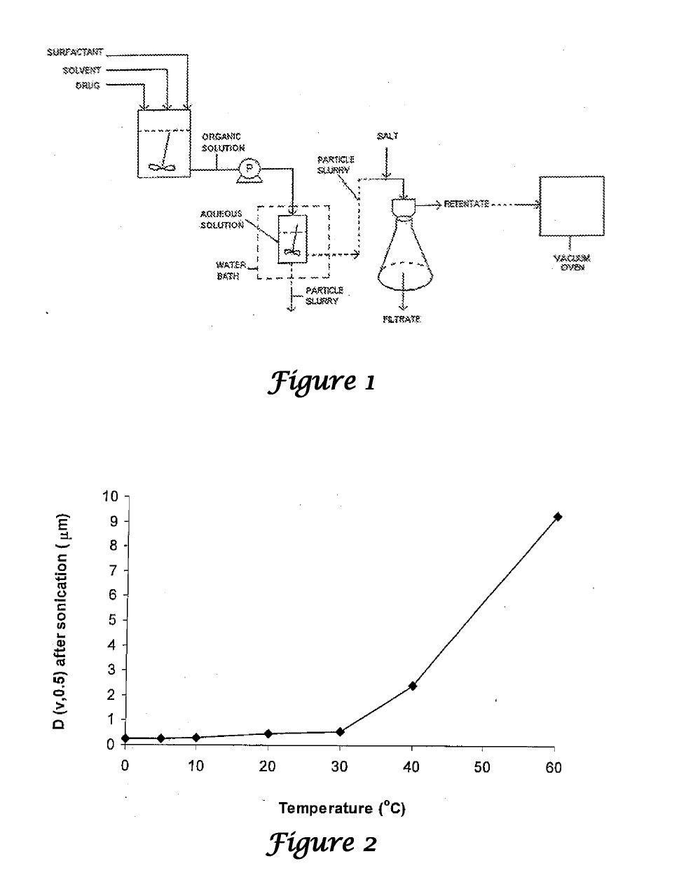

A schematic of the antisolvent precipitation followed by the flocculation method is shown in FIG. 1. The naproxen solution in methanol or ethanol was fed by a HPLC pump through a 1 m long 1/16 in. o.d.×0.030 in. i.d. stainless steel tube. The organic solution was sprayed with a jet in the shape of a cylindrical column without atomization through the stainless steel tubing into pre-cooled aqueous surfactant solution. The aqueous surfactant solution was contained in a 250 ml glass cylinder submerged in a water/ethylene glycol bath controlled to 3° C. The tip of the stainless steel tube was submerged approximately 4 cm under the surface of the aqueous solution. To enhance the mixing between organic phase and aqueous phase, a magnetic stir bar was placed inside the glass cylinder and stirred at a fixed rate to form a vortex. Unless specified elsewhere, 7% w/v naproxen with or without surfactant was typically dissolved into methanol. The organic solution was then sprayed into 50 ml aqueous solution at 5 ml/min for 1.4 min to yield a suspension concentration of 10 mg/ml. After spraying for a required time to produce the desired suspension concentration, the suspension was analyzed within 5 min to determine the particle size by static light scattering with Malvern Mastersizer-S (Malvem Instruments Ltd.). The suspension was sonicated in the Mastersizer to break up the aggregates until a stable particle size distribution was obtained. The particle size of the same suspension was also measured after 5 minutes sonication with a powerful sonicator (Branson Sonifer 450, Branson Ultrasonics Corp.) in an ice/water bath at an output control of 7 and 30% duty cycle.

Recovery of naproxen nanoparticles by salt flocculation followed by filtration and drying is depicted in FIG. 1. After particle size measurement, the suspension produced was sonicated in an ice/water bath at an output control of 7 and 30% duty cycle for 5 minutes. Then, a given volume of 20% w/v sodium sulfate was added into the suspension and mixed thoroughly with a spatula. The suspension was left at room temperature for 3 min to form large flocs. The flocs were filtered with P2 filter paper (Fisher Scientific) under vacuum (−27 in. Hg). Gentle stirring was necessary in the first 3 min of filtration to prevent forming a dense precipitate cake, which would increase resistance to filtration and result in a long filtration time. The filtration was continued for 10s after no more water droplets were formed at the tip of the ceramic funnel The precipitate was placed into a vacuum oven and dried overnight at room temperature at a vacuum of −30 in. Hg. To check the reproducibility of this process, for each formulation, triplicate samples were prepared. The precipitate weight was determined after vacuum drying. A known amount of this dry powder, about 5 mg, was dissolved into 50 ml acetonitrile/water mixture, 50:49 (v:v). The potency of naproxen was determined by measuring the naproxen concentration by HPLC (Shimadzu, LC-IOAT VP, Japan). The salt concentration was determined from the electrical conductivity as described below. The surfactant composition in the final powder was calculated by difference given the total precipitate weight, the drug potency and the salt concentration. The drug recovery was calculated from the HPLC measurement and the total amount of drug fed to the suspension in the antisolvent process. The surfactant recovery was calculated with the same method, given the surfactant composition in the dry powder. Wide angle X-ray scattering was employed to detect the crystallinity of naproxen. CuK al radiation with a wavelength of 1.54054 A at 40 kV and 20 mA from a Philips PW 1720 X-ray generator (Philips Analytical Inc., Natick, Mass.) was used. The samples were well mixed to minimize the effects of preferred orientation. The reflected intensity was measured at a 20 angle between 5 and 45° with a step size of 0.05° and a dwell time of 1 s.

Particle size distributions based on volume fraction were measured for the original antisolvent suspension prior to flocculation and for the dried powders after redispersion and sonication with laser light scattering (Mastersizer-S, Malvern Instruments Ltd., U. K.). Approximately 5 ml of the suspension with a concentration of 10 mg drug/ml water was diluted with 500 ml distilled water, to produce a light obscuration in the desired range of 10-30%. The amount of drug in the water was several folds above the solubility limit. In control demonstrations, samples were stirred in the Mastersizer for up to 10 minutes, and there was very little change in the particle size distribution. To illustrate the redispersibility of the dry powders, about 100 mg dry powder was suspended into 500 ml distilled water to produce an obscuration in the range 10-30%. After 1 min, the particle size distribution was measured. Ultrasound was used in the measurement to break up any aggregated particles. The uncertainty in mean particle size for different sprays at the same demonstration conditions was about 10 to 15%.

The residual concentration of sodium sulfate was measured by conductivity. The conductivities of a series of standard concentration of sodium sulfate solutions in acetonitrile/water mixture (v/v=50:50) were measured with a conductivity probe with cell constant of 1/cm (Model 3252, YSI Inc., Yellow Springs, Ohio). For salt concentrations ranging from 0.003 to 0.05 mg/ml, a linear standard line was obtained with a correlation coefficient of 0.9999. A small amount, about 5 mg, of dried naproxen powder was dissolved in the same acetonitrile/water mixture to yield a salt concentration in the linear range of the standard curve to determine the conductivity.

Optical microscopy and scanning electron microscopy (SEM) were used to visualize the morphology of the naproxen flocs in the suspension and in the dried powder. A drop of antisolvent suspension flocculated with sodium sulfate solution was placed onto a microscope slide (25×75 mm, Erie Scientific Co.) and carefully covered with a cover glass (22×22 mm, Fisher Scientific, Pittsburg, Pa.). The morphology of naproxen flocs in the suspension was examined with an optical microscope (Axioskop 2 plus, Cal Zeiss Vision GmbH, Germany). Another suspension produced with the same demonstration conditions was filtered with a P2 filter paper under vacuum for 11 min. The dried powders were mounted on an aluminum cylinder using double adhesive carbon conductive tabs (Ted Pella Inc.) and coated with Au for 25s using a Pelco Model 3 sputter-coater under an Ar atmosphere. A Hitachi S-4500 scanning electron microscope (SEM) (Hitachi Instruments Inc.) at an accelerating voltage of 101V with a secondary electron detector was used to obtain digital images of the samples.

The dissolution of naproxen powder was tested in pure water using a USP Apparatus II (Vanke17000, Vankel Technology) at 50 rpm. All dissolution tests were conducted at sink conditions, in this case at 15% of its solubility in water. Antisolvent powders containing approximately 2 mg naproxen were added to 900 ml pure water at 37° C. Aliquots of the dissolution medium (5 ml) were sampled at 2, 5, 10, 20 and 30 minutes. The aliquots were filtered through a 0.45 μm syringe filters and 2 ml of each sample was diluted with 0.1 ml acetonitrile before analysis. Naproxen concentrations were measured using HPLC (Shimadzu, LC-IOAT VP, Japan). Dissolutions were repeated in duplicate or triplicate for the powder aliquots, and the average deviations are reported in the dissolution figures.

B.P. grade itraconazole (Itz) was purchased from Hawkins, Inc. Hydroxypropyl methylcellulose E5 grade (HPMC) (viscosity of 5 cP at 2% aqueous 25° C. solution) was a gift from The Dow Chemical Corporation. Poly(ethylene oxide-b-propylene oxide-b-ethylene oxide) (P407) with a nominal molecular weight of 12,500 and a PEO/PPO ratio of 2:1 by weight was purchased from Spectrum Chemical Manufacturing Corporation. Stabilized p.a. grade 1,3-dioxolane was obtained from Acros Organics. HPLC grade acetonitrile (ACN), A.C.S. grade hydrochloric acid (HCl), diethanolamine (DEA), sodium dodecyl sulfate (SDS), and sodium sulfate anhydrous (Na₂SO₄) were used as received from Fisher Chemicals. A glass transition temperature (T_{g}) of 154° C. has been reported for HPMC E5 and estimated as −21° C. for P407. Type P2 filter paper with an area of 95 cm² and an estimated pore size of 1-3 μm was purchased from Fisher Scientific.

Using an antisolvent precipitation method, nanoparticle dispersions of Itz were produced at about 3° C. Deionized water (50 mL) containing an appropriate quantity of HPMC was used as the antisolvent phase into which 15 g of 1,3-dioxolane containing 3.3% (wt) Itz (and P407 in some cases) was injected using a 19G needle in approximately 6 s to form a fine precipitate. A control demonstration was performed in which the nanoparticles were recovered by rapid freezing and lyophilization. For this rapidly frozen control, the organic phase was first separated from the aqueous dispersion via vacuum distillation at 40 torr and 38° C. The aqueous dispersion was added dropwise to liquid nitrogen and lyophilized to form a powder using a Virtis Advantage Tray Lyophilizer (Virtis Company) with 24 hr. of primary drying at −35° C. followed by 36 hours of secondary drying at 25° C. Unless noted otherwise, the particle size distributions of the original dispersion and the redispersed flocs were determined by static light scattering using a Malvern Mastersizer-S (Malvern Instruments Ltd.)

In a separate control study, after vacuum distillation the aqueous dispersion was spray dried using a Buchi mini-spray dryer Model 190 (Brinkmann Instruments Co.) equipped with a 0.7 mm diameter two-fluid nozzle. Compressed air at 120 psi was used for the atomizing nozzle, with the flow rate controlled to 200 mL/sec. The dispersion was fed at a rate of 5 mL/min. An inlet temperature of 140° C. and outlet temperature of 90° C. were maintained throughout the process.

Dispersions were heated after organic solvent removal either rapidly to 98° C., or slowly to 92° C. For rapid heating, the dispersion was injected at 10 mL/min through about 3′ of 0.03″ ID stainless steel tubing that was heated by a 98° C. water bath. Upon exiting the heat exchanger, the dispersion was immediately quench-cooled into water (3° C.). For slow heating, the dispersion was placed in a beaker, which was heated at a rate of 2.5° C./min. The dispersion was allowed to remain at 92° C. for 10 minutes before quench cooling in an ice bath. In both cases, particle size measurement was taken immediately after quench cooling using dynamic light scattering.

After particle size determination, the nanoparticle dispersions were flocculated by addition of a 1.5M solution of Na₂SO₄ at a volume ratio of 12:5 (salt solution/suspension). This approach produced a SO₄⁻² concentration of 1 M in the final mixture. Immediately after salt addition, the flocs took up the entire aqueous volume, as seen in FIG. 1. The flocculated suspension was stored at room temperature for 3 minutes during which larger flocs formed. The flocs were filtered with 11 cm diameter P2 filter paper under vacuum. The filtration was continued until no water could be observed on top of the filter cake, typically after <8 minutes. A 30 ml aliquot of HPMC aqueous solution (identical to the antisolvent phase of the sample) was chilled in an ice bath and used to rinse the filter cake immediately after filtration. The filter cake was then allowed to dry at room temperature and atmospheric pressure overnight. Dried powders were obtained by gently scraping the filter paper.

High Performance Liquid Chromatography (HPLC): Solutions from dissolution illustrations and dissolved dried powders were analyzed for drug concentration using a Shimadzu LC-10 liquid chromatograph (Shimadzu Corporation, Columbia, Md.) with an Alltech 5 μm Inertsil ODS-2 C18 reverse-phase column (Alltech Aassociates, Inc.). The mobile phase was ACN/water/DEA 70:30:0.05 (volume ratio) and the flow rate was 1 ml/min. Using a detection wavelength of 263 nm, the Itz peak eluted at 5.4 minutes. The standard curve linearity was verified from 500 to 1 mg/ml with an r² value of at least 0.999.

In order to determine the quantity of residual salt in the final filter cake, approximately 15 mg of dried powder was dissolved in 100 mL of 50:50 ACN/water. Using a YSI 3100 conductivity instrument (YSI Inc.), the conductivity of the solution was determined and compared to a standard curve of Na₂SO₄ for concentrations from 0.004 to 0.137 mg/mL. The standard curve was linear with an r² value of 0.9998. The drug concentration of the solution was determined by HPLC and used to calculate the drug loading of the final powder.

Approximately 50 mg of dried powder was compacted with about 500 kg using a Model M Carver Laboratory Press (Fred S. Carver, Inc.) to form a flat sided pellet. A 10 μL drop of pH 6.8 phosphate buffer without SDS was placed on the pellet, and the contact angle was measured within 15 sec. using a Model 100-00-115 Goniometer (Rame-Hart Inc.). Measurements were made in quadruplicate and the average and standard deviation were reported.

Dried powders were redispersed in pure water at a concentration of approximately 4 mg/mL. Using a Branson Sonifier 250 (VWR Scientific), the dispersion was sonicated for 2 minutes at 50% duty cycle. The aqueous dispersions were then flash frozen onto aluminum SEM stages maintained at −200° C. with liquid nitrogen. After lyophilization to remove the water, the remaining particles were gold-palladium sputter coated for 1 minutes prior to analysis on a Hitachi S-4500 field emission scanning electron microscope at an accelerating voltage of 15 kV.

Drug powders were placed in hermetically sealed aluminum pans and scanned using a 2920 modulated DSC (TA Instruments) with a refrigerated cooling system. The samples were purged with nitrogen at a flow rate of 150 mL/min. The amplitude used was 1° C., the period 1 minutes, and the underlying heating rate 5° C./minute. Both forward and reverse heat flow were used to characterize the thermal transitions of samples.

Solubilities of metastable solutions of amorphous drug and rates of supersaturation were measured in pH 6.8 phosphate buffer made by mixing 1 part 0.1N HCl with 3 parts 0.2M tribasic sodium phosphate at 37.2° C. In the media with 0.17% (wt./vol) SDS, the equilibrium crystalline solubility was 14 μg/mL, as measured in triplicate similarly to the method described in a previous demonstration. A USP paddle method was adapted to accommodate the small sample sizes using a VanKel VK6010 Dissolution Tester with a Vanderkamp VK650A heater/circulator (VanKel). Dissolution media (50 mL) were preheated in small 100 mL capacity dissolution vessels (Varian Inc.). A sample weight (about 17.5 mg drug) equivalent to approximately 25-times the equilibrium solubility of ITZ in pH 6.8 media containing SDS was added. Sample aliquots (1.5 mL) were taken at various time points. The aliquots were filtered immediately using a 0.2 mL syringe filter and 0.8 mL of the filtrate was subsequently diluted with 0.8 mL of ACN. In all cases, the filtrate was completely clear upon visual inspection and dynamic light scattering of the filtrate gave a count rate of less than 20 kcps (too small for particle size analysis). For all samples, the drug concentration was quantified by HPLC. The supersaturation levels are reported as the drug concentration divided by the crystalline solubility. Supersaturation was plotted versus time and the area under the curve (AUC) was calculated using the numerical integration.

The effect of temperature on the particle size distribution in the aqueous suspensions produced by antisolvent precipitation was determined 5% w/v naproxen and 2% w/v poloxamer 407 in ethanol solution was sprayed into 50 ml 3% w/v PVP K-15 at flow rate of 1 ml/minute for 5 minutes. The final suspension concentration was 5 mg/ml. The organic solution was at room temperature. As shown in FIG. 2, the mean particle size doubled from 270 nm at 0° C. to 540 nm at 30° C. At temperatures higher than 30° C., the mean particle size increased markedly and reached 9.3 μm at 60° C. This temperature behavior was also observed for ketoconazole, another poorly water-soluble drug. When 4% w/v ketoconazole and 2% w/v poloxamer 407 in methanol solution was sprayed into 50 ml 3% w/v PVP K-15 at flow rate of 1 ml/min for 20 min, to form a suspension concentration of 16 mg/ml, the mean particle size of ketoconazole increased from 350 nm at 4.6° C. to 9.1 μm at 23.8° C. Several factors may lead to larger particles at high temperature, including degree of supersaturation, nucleation rate, crystal growth rate, colloidal interactions between surfactant stabilizers and Ostwald ripening. We simply list these factors; a great deal of work would be needed to sort out the magnitude of each effect. The solubility of both drugs in the organic-water mixture increases with temperature. Thus, for a given drug concentration in the feed, the supersaturation decreases with temperature. The lower supersaturation lowers the nucleation rate. The smaller number of nuclei may be expected to produce larger particles for a given drug concentration in the final suspension. Also, the diffusion rate of drug molecules to the surface of the growing particles and the kinetics of addition of drug molecules to the surface increase with temperature. Furthermore, Ostwald ripening or diffusion of molecules from smaller crystals to larger crystals is also favored by higher solubility at higher temperature. The solvation of ethylene oxide groups in the poloxamer tails is more efficient as temperature decreases due to hydrogen bonding between water and the ether oxygen. Greater solvation will improve the repulsive forces between ethylene oxide blocks on approaching particles to improve steric stabization. Based on these demonstrations, all other demonstrations were performed at or below room temperature.

As shown in Table 1, four systems were demonstrated. In system A, only 10% w/v PVP K-15 was used in the organic phase and there was no surfactant in the aqueous phase. This system was designed for long-term stability since PVP K-15 has a high glass transition temperature which enhances drug stability by decreasing the mobility of drug molecules. In system B, 10% w/v poloxamer 407 was used in the organic phase, as it has been shown to be an effective stabilizer and 3% w/v PVP K-15 was included in the aqueous phase to raise the T, of the final powder. To attempt to raise the potency of the drug, in system C the poloxamer 407 concentration was reduced to 5% w/v and in system D, the PVP IC-15 concentration was reduced to 1% W/V. The overall drug/poloxamer 407RVP K-15 ratios in the final suspension based on the amounts fed from both organic and aqueous phase during spray time are also given. The total solid weight and total drug potency (weight of drug/total weight excluding salt) are listed for each system.

TABLE 1
Systems investigated.

| | | | Drug/ | | |
|---|---|---|---|---|---|
| | | | Pluronic/ | Total | Original |
| | | | PVP ratio | solid | drug |
| | Organic | Aqueous | in the | weight | loading |
| | phase | phase | suspension | (g) | (%) |
| A | 10% PVP K-15 | Pure water | 1/0/1.4 | 1.22 | 41.2 |
| B | 10% Pluronic F127 | 3% PVP K-15 | 1/1.4/3 | 2.72 | 18.5 |
| C | 5% Pluronic F127 | 3% PVP K-15 | 1/0.7/3 | 2.36 | 21.3 |
| D | 5% Pluronic F127 | 1% PVP K-15 | 1/0.7/1 | 1.36 | 37.0 |

Particle size of naproxen in aqueous suspensions: As shown in Tables 2 and 3, aggregated naproxen nanoparticles were produced during antisolvent precipitation at both 22° C. and 3° C. With only PVP K-15 in the organic phase and pure water in the aqueous phase (System A, overall drug/poloxamer 407RVP K-15 ratio=1:0:1.4), the particle sizes were large in the original suspensions after spraying at either 22° C. or 3° C. Even after sonication in the laser light scattering chamber, the mean particle size was still undesirably large, as shown in Table 2. After the original suspensions were stirred with a magnetic stir bar for 20 h at room temperature and then sonicated, the mean particle sizes became extremely small, well below 0.4 μm at both temperatures. More surfactant may adsorb to the particles during stirring and aid breakup of the aggregates.

TABLE 2

| Temperature | | | | | |
|---|---|---|---|---|---|
| T = 22° C. | | | T = 3° C. | | |
| suspension | | | | | |
| | D(v, 0.1/0.5/0.9) | Son. | | D(v, 0.1/0.5/0.9) | Son. |
| D(v, 0.1/0.5/0.9) | after sonication | time | D(v, 0.1/0.5/0.9) | after sonication | time |
| w/o sonication (μm) | with Mastersizer (μm) | (min) | w/o sonication (μm) | with Mastersizer (μm) | (min) |

Aqueous phase: DI water

| | | | | | | |
|---|---|---|---|---|---|---|
| Original | 7.5/15.9/28.4 | 2.4/5.3/7.2 | 7 | 6.2/12.6/153.1 | 2.3/4.5/6.3 | 15 |
| suspension | | | | | | |
| After 20 hours | 4.5/6.4/11.0 | 0.15/0.36/2.1 | 3 | 6.1/12.1/19.7 | 0.11/0.29/0.88 | 5 |
| stirring at room | | | | | | |
| temperature | | | | | | |

Aqueous phase: 5% w/v Pluronic F127

| | | | |
|---|---|---|---|
| Original suspension | 6.9/15.9/42.4 | 0.10/0.29/3.3 | 6 |

TABLE 3
tems

| D(v, 0.1/0.5/0.9) | D(v, 0.1/0.5/0.9) | V of | Cₛₐₗₜ | D(v, 0.1/0.5/0.9) | D(v, 0.1/0.5/0.9) | | Release |
|---|---|---|---|---|---|---|---|
| in the original | after 5 min | salt | in susp. | after redispersion | after redispersion | t_{son.} | in 2 min |
| suspension (μm) | sonication (μm) | (ml) | (M) | into water (μm) | and sonication (μm) | (min) | (%) |
| 4.5/6.4/11.0 | 0.15/0.3/2.1 | 175 | 1.10 | 16.5/91.6/243.0 | 0.12/0.32/3.24 | 3 | 27.3 |
| | | 200 | 1.13 | 29.4/155.7/447.1 | 0.22/2.02/11.6 | 6 | 25.5 |
| 0.14/0.48/95.2 | 0.11/0.29/1.01 | 100 | 0.94 | 0.10/0.30/6.8 | 0.10/0.27/3.07 | 1 | 95.4 |
| | | 125 | 1.01 | 0.11/0.31/1.87 | 0.11/0.28/1.13 | 1 | 96.4 |
| | | 150 | 1.06 | 0.23/0.48/19.3 | 0.11/0.32/7.72 | 1 | 74.8 |
| 0.11/0.30/3.8 | 0.12/0.29/0.68 | 125 | 1.01 | 0.11/0.31/2.57 | 0.10/0.25/0.79 | 1 | 94.9 |
| 0.14/9.8/173.9 | 0.10/0.29/6.95 | 125 | 1.01 | 0.28/2.65/22.3 | 0.19/0.38/4.10 | 2 | 49.5 |

In order to attempt to reduce the aggregation of the particles at 3° C., 5% w/v poloxamer 407 was added to the aqueous phase to modify system A. The mean particle size of the original suspension was 15.9 μm. After 6 minutes sonication in the Mastersizer, the mean particle size decreased to 0.29 μm. In this case the aggregates broke up into small particles without the need to stir the solution for 20 hours. Since the copolymer poloxamer 407 has hydrophobic moieties which adsorb onto hydrophobic drug particle surfaces and two separate hydrophilic blocks, the adsorption of poloxamer 407 with PVPK-15 may be expected to provide greater steric repulsion 1 and looser aggregates. The looser aggregates may be broken up more easily by sonication on the basis of the smaller particle sizes.

The particle sizes for the formulations in addition to system A are presented in Table 3. The results for the suspensions prior to flocculation are shown in the second and third columns. In order to avoid the need for stirring for long periods of time to break up aggregates, the organic phase stabilizer was changed to poloxamer 407 for all of the systems in Table 1 except system A. In addition, PVP K-15 was utilized in the aqueous phase to achieve a sufficiently high Tg, for the final powder. For system B and C, the mean particle sizes were extremely small, below 0.5 μm, in the original suspension even without sonication. After sonication, the particle size decreased only a small amount. However, when the PVP K-15 concentration in the aqueous phase was lowered to 1% w/v in system D, the mean particle size of the original suspension increased to 9.8 μm. These aggregates readily broke up upon sonication. After sonication the mean particle size was 0.29μ for systems B-D, although the D (v, 0.9) was 7.0 μm for system D. Without sonication, a higher concentration of 3% w/v PVP K-15 in the aqueous phase was helpful in forming the nanosuspension. In system B and C, the overall concentration of PVP K-15 was much higher than that of poloxamer 407, unlike the case for system D. The higher overall stabilizer concentration may have been required to provide enough steric stabilization to prevent aggregation of naproxen nanoparticles.

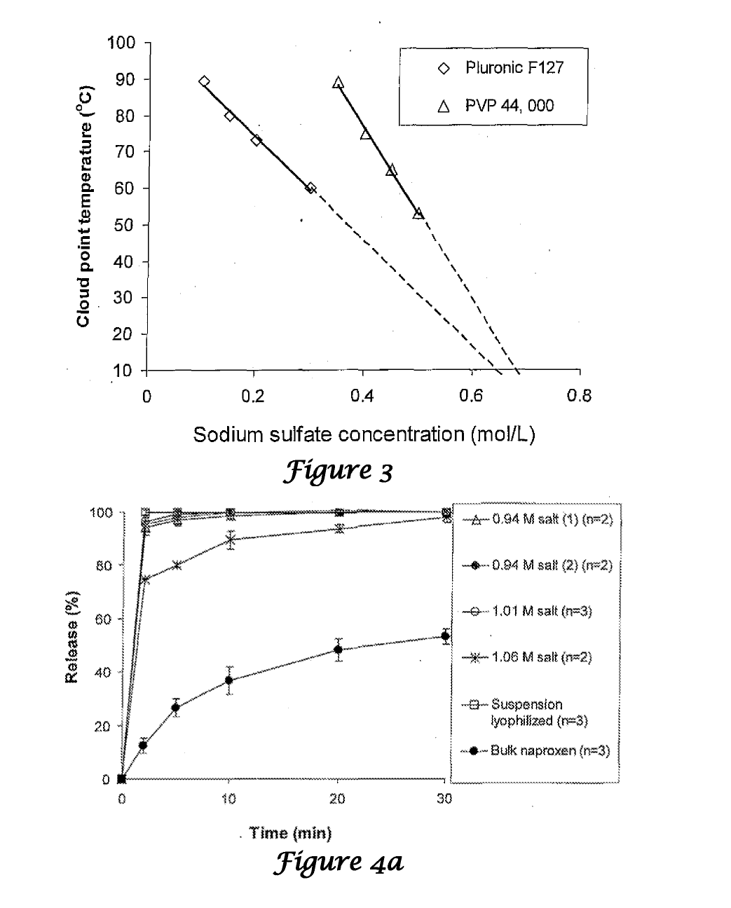

Polymer-salt cloud point concentrations and flocculation of naproxen nanoparticles with various salts. Sterically stabilized dispersions may be flocculated by reducing the solvency of the dispersion medium for the stabilizing moieties to induce the onset of instability. Pelton reported that the critical flocculation temperature (CFT) corresponds to the cloud point temperature of the stabilizing polymer. A few reports have described the influence of inorganic salts on the cloud point behavior of poloxamers and PVP. As shown in FIG. 3, the cloud point temperatures of PVP 44,000 and poloxamer 407 decrease linearly with increasing concentrations of sodium sulfate. At a given temperature, the polymer precipitates as the salt concentration increases. In FIG. 3, the concentration of PVP was 0.74% w/v on the basis of a partial specific volume of 0.952 cm³/g for PVP. The concentration of poloxamer 407 was 5 mg/ml or 0.5% w/v. PVP contains N—C═O units on the lactam rings that hydrogen bond with water as observed with viscometric measurements and spectrophotometric demonstrations. The decrease of the Huggins constant with the addition of inorganic salt into PVP aqueous solution indicates a loss in water-PVP H-bonding, and thus in PVP hydration, leading to precipitation. For the same reasons, salt also precipitates PEO homopolymers and of PEO-containing nonionic surfactants such as poloxamers.

The reciprocal of cloud point temperatures, 1/Tcp, of two fractionated and two unfractionated PVP samples have been shown to be linear in 1M_{w}. With a lower molecular weight of 10,000, more sodium sulfate is required to precipitate the PVP K-15 used in this demonstration than for the PVP 44,000 shown in FIG. 3.

The stabilizing moieties collapse at the onset of the cloud point where the solvent is worse than a θ-solvent, as a result of the large number of interactions of the hydrophilic groups. Steric repulsion becomes weak and the stabilizing chains interact with each other leading to sticky Brownian collisions and flocculation. Some free surfactant might also precipitate out from the solution and interact with flocculating particles. Since the particle size of coated naproxen was typically larger than 200 nm and the molecular weight of stabilizing polymers was not more than 12,500 Da, the steric stabilization of the polymer chains does not completely screen the van der Waals attraction between the particles. If the flocs are sufficiently large, the drug particles may easily be recovered from the solution by filtration.

To more fully understand the effect of salinity of flocculation, the critical flocculation salinity was measured for a 1.26% w/v concentration of either PVP K-15 or poloxamer 407. The solvent was a mixture of methanol (12.6%, v/v) and water (87.4%, v/v) with the same composition as the system A suspension. A concentration of 1.06 M salt was needed to precipitate PVP from solution at room temperature in the methanol/water mixture while only 0.98 M was needed without any methanol. For pure poloxamer 407, 0.71 M salt concentration was needed for solution with methanol, while 0.58 M was need without methanol. Apparently, at the same concentration and temperature, poloxamer 407 was precipitated much more easily with sodium sulfate than was PVP K-15, which is consistent with FIG. 3. Less salt is needed to flocculate poloxamer 407 than PVP (Mw=44,000) at 25° C. from pure water without methanol. This difference is expected to be even larger for PVP K-15 since a greater salt concentration is required to precipitate the lower molecular weight. The critical flocculation salinities, despite the presence of methanol are in the same order expected from the cloud points. Methanol also raises the cloud point temperature for poloxamine 908, a copolymer containing polyethylene oxide moieties.

A much lower salt concentration is required for a divalent sulfate to precipitate these polymers relative to a monovalent anion. Three multivalent inorganic salts which are known to produce a large reduction in the cloud point temperature of PVP and poloxamer 407, sodium carbonate, sodium sulfate and sodium phosphate, were used to attempt to flocculate the nanoparticles. Large flocs were formed with sodium sulfate, and they were readily filterable. 20% w/v sodium carbonate and 10% w/v sodium phosphate were also tested for flocculation. In each case, when a small volume of the salt solution was added to the suspension to produce a molarity less than 0.04 M, a clear solution was formed as the naproxen particles dissolved. When 0.4 ml sodium carbonate or 1 ml sodium phosphate solution was added into 25 ml of the suspension, the pH of the solution increased to 11.3 for CO₃²⁻ and 11.9 for PO₄³⁻ due to the acid/base hydrolysis. The solubility of naproxen increases from 0.0159 mg/ml in pure water to 196.7 mg/ml at a pH higher than 8, which is well above the pKa, of naproxen. This solubility is much higher than the naproxen concentration in the suspension, consistent with dissolution observed. Based on these results all of the flocculation demonstration utilized sodium sulfate.

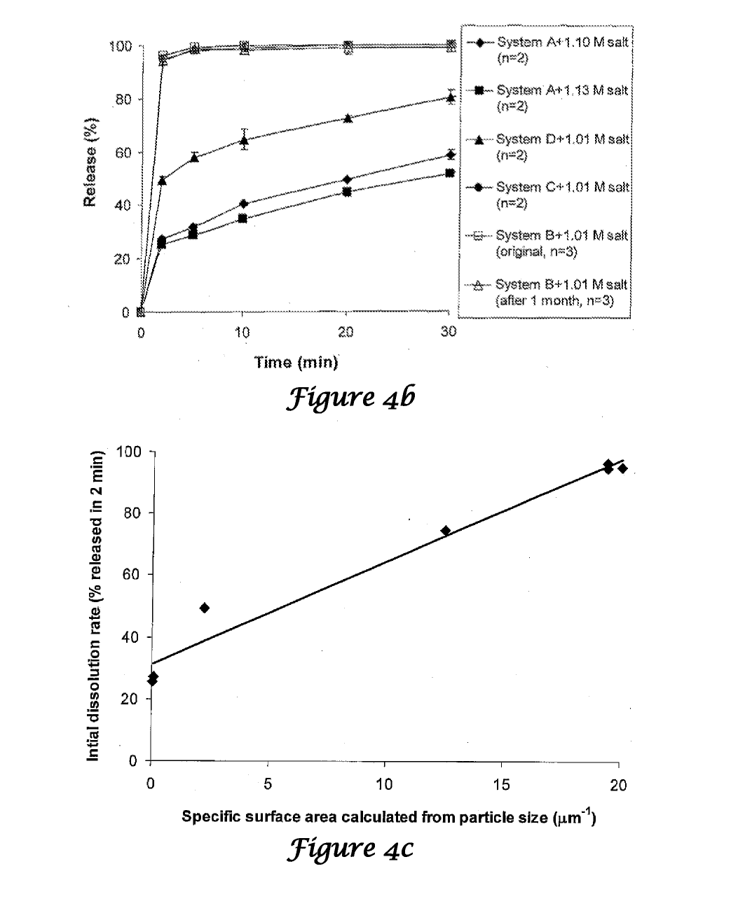

The dissolution rates of the dried naproxen powders are shown in FIG. 4 (a) for System B and for a series of formulations in FIG. 4(b). The dissolution rates were reproducible as shown by the error bars which represent the average deviation, defined by (1/n)Σ|x−x|. The dissolution rates were very high for systems B and C, and significantly slower for systems A and D. As shown in FIG. 4 (a), approximately 100% naproxen was released in 2 min for the suspension dried by lyophilization. For samples flocculated with 100 ml or 125 ml salt solution, the dissolution rates of naproxen were only slightly slower than for the lyophilized ones. The flocculated sample of system B was stored with desiccant under vacuum at room temperature. After 1 month, the dissolution rate of this sample did not change, as shown in FIG. 4 (b). The differences in these dissolution rates will now be analyzed in terms of the particle size distributions. Particle redispersibility into water after flocculation was followed by filtration and vacuum drying. The dry powder was redispersed into pure water, and the particle sizes are shown in the sixth and seventh columns of Table 3. For system A flocculated with a salt concentration of 1.10 M or 1.13 M, the dried naproxen particles formed a very coarse suspension upon stirring without sonication. The mean particle size of the dried particles was 91.6 μm without sonication at 1.10 M salt concentration, but decreased to 0.32 μm after 3 min sonication. With a higher salt concentration of 1.13 M, The mean particle size of naproxen decreased from 155 μm without sonication to 2.02 μm after 6 minutes sonication. Because the mean particle size was only 0.29 μm after flocculation and filtration but before drying, the particle growth must have taken place during the drying step. The slow dissolution rates in system A are consistent with the large particle sizes upon redispersion without sonication.

In order to illustrate the reversibility of flocculation, a suspension flocculated at 1.13 M salt concentration was filtered with a 0.45 μm WHATMAN® nylon membrane filter paper (Whatman International Ltd.) for 43 minutes The particle size of redispersed naproxen after drying was 17.1 μm after sonication. Apparently, if the particles are allowed to remain flocculated for more than 40 minutes, redispersion is no longer complete. Perhaps slow diffusion of the stabilizer away from the stress zones that are created by the close approach of the particles leads to irreversible aggregation.

For system B, with either 0.94 M or 1.01 M salt, the dried particles could be redispersed into water readily with particle sizes similar to the original suspensions. Consequently the dissolution rates were extremely rapid with 96% release in two minutes As the salt concentration was increased to 1.06 M, the mean size of redispersed particles increased modestly to 0.48 μm with a high D (v, 0.9) of 19.3 μm. Likewise, the dissolution rate decreased to 75% in two minutes. Here, 1 min sonication was needed to reduce the particle size to the value prior to flocculation. Higher salt concentrations appeared to produce tighter flocs after drying, requiring sonication to fully break them up, whereas sonication was not needed for the lower salt concentrations.

For system C, particles flocculated at 1.01 M salt concentration could be redispersed easily after drying to achieve a similar particle size as in the original dispersion. Consequently, the dissolution rate was extremely fast as in the case for the two lowest salinities in system B. However, for system D, with a low PVP K-15 concentration in the aqueous phase, the mean particle size of the redispersed dried powders without sonication was extremely large, 2.7 μm. The size decreased most of the way to the value prior to flocculation after 2 minutes sonication. The dissolution rate was much slower than in systems B and C. Therefore, the dissolution rate is more closely related to the aggregate size than the size of the particles after sonication.

For all of the examples in Table 3, the dissolution rate was correlated closely with the particle size of the redispersed powder without sonication. Assuming spherical particles, the specific surface area of naproxen particles was calculated by, S=6/D, where D is the average diameter of the particles from light scattering shown in the sixth column of Table 3. As shown in FIG. 4 (c), a straight line with a correlation coefficient of 0.97 was obtained between initial dissolution rate (% released in 2 minutes) and specific surface area of naproxen particles after redispersion. This correlation is consistent with Noyes-Whitney equation, where the dissolution rate is proportional to the specific surface area of drug particles.

The reproducibility of the salt flocculation process on precipitate weight, drug potency, salt concentrations, surfactant concentration, drug and surfactant yield was also investigated. The filtration time and properties of the precipitate including naproxen potency (drug/total solids, w/w), drug yield (fraction in precipitate versus total amount fed), surfactant yield (fraction in precipitate) and filtration selectivity ((g precipitate drug/g filtrate drug)/(g precipitate surfactant/g filtrate surfactant)) for four systems flocculated with various concentrations of sodium sulfate are shown in Table 4. Compared to the overall naproxen potencies in Table 1 for the suspensions, the naproxen potencies in the precipitate were higher, even though Table 1 did not include salt, since some free or non-adsorbed surfactant was removed with the filtrate. For system A, even at a salt concentration of 1.10 M, the resulting flocs were not stable. Stirring or pouring broke up the fragile flocs. Thus, the precipitate weight was not reproducible, relative to the other systems. The poor flocculation was due to the salinity too close to the critical flocculation salinity, 1.06 M with 12.6% v/v methanol in the suspension. When salt concentration was increased to 1.13 M, larger flocs formed and consequently the drug and surfactant yields increased. The naproxen potencies were similar for both salinities and were well above the overall potency in the suspension prior to flocculation.

TABLE 4

| | Cₛₐₗₜ | Filtration | Precip. | Drug | Salt | Surf. | Drug | Surf. | |
|---|---|---|---|---|---|---|---|---|---|
| | in susp. | time | weight | Potency | conc. | conc. | yield | yield | selectivity |
| System | (M) | (min) | (g) | (%) | (% w/w) | (% w/w) | (%) | (%) | for filtration |
| A | 1.10 | 8.6 | 0.5083 ± 0.0994 | 69.0 ± 0.2 | 25.2 ± 0.3 | 5.8 ± 0.4 | 70.2 ± 14.0 | 4.4 ± 0.2 | 94.5 ± 73.9 |
| | | | (n = 3) | (n = 3) | (n = 3) | (n = 3) | (n = 3) | (n = 3) | |
| A | 1.13 | 7.7 | 0.6552 ± 0.0030 | 66.3 ± 0.7 | 19.6 ± 0.7 | 14.1 ± 1.4 | 86.9 ± 1.3 | 12.9 ± 1.2 | 45.5 ± 6.8 |
| | | | (n = 2) | (n = 2) | (n = 2) | (n = 3) | (n = 3) | (n = 3) | |
| B | 0.94 | 16.5 | 1.0768 ± 0.0596 | 38.2 ± 0.8 | 10.6 ± 1.4 | 51.2 ± 1.1 | 79.7 ± 1.4 | 24.1 ± 0.9 | 12.4 ± 0.5 |
| | | | (n = 3) | (n = 3) | (n = 3) | (n = 3) | (n =3) | (n = 3) | |
| B | 1.01 | 11 | 1.2523 ± 0.0113 | 36.7 ± 0.7 | 10.5 ± 0.5 | 52.9 ± 0.5 | 91.8 ± 1.4 | 29.9 ± 0.3 | 28.8 ± 8.5 |
| | | | (n = 4) | (n = 4) | (n = 4) | (n = 3) | (n = 3) | (n = 3) | |
| B | 1.06 | 11.5 | 1.4825 ± 0.0139 | 31.8 ± 0.7 | 9.3 ± 0.5 | 58.9 ± 1.0 | 94.1 ± 1.7 | 39.4 ± 1.0 | 28.9 ± 12.3 |
| | | | (n = 3) | (n = 3) | (n = 3) | (n = 3) | (n = 3) | (n = 3) | |
| C | 1.01 | 12 | 0.9021 ± 0.0017 | 54.1 ± 0.5 | 8.8 ± 0.0 | 37.1 ± 0.7 | 97.6 ± 1.5 | 18.0 ± 0.3 | 316.1 ± 206.5 |
| | | | (n = 2) | (n = 2) | (n = 2) | (n = 2) | (n = 2) | (n = 2) | |
| D | 1.01 | 14 | 0.8930 | 55.7 ± 0.5 | 6.4 ± 0.2 | 37.9 | 99.4 | 39.6 | 256.8 |
| | | | | (n = 2) | (n = 2) | (n = 2) | (n = 2) | (n = 2) | |

For system B, a control was performed in which the suspension was filtered without adding any salt. Without flocculation by salt, only 0.050 g precipitate was recovered as the particles were too small for the filter. At salt concentration of 0.47 M, only a small amount of flocculation occurred, and the drug particles plugged up the filter paper in 5 minutes. As the salt concentration increased from 0.94 to 1.01 to 1.06 M, the drug and surfactant yields increased significantly. A drug yield higher than 92% was obtained at a salt concentration higher than 1.4 times the critical flocculation salinity of poloxamer 407. The naproxen potency decreased a small amount with salinity and was about double the overall potency. The relative deviation (average deviation/mean value) of the precipitate weight decreased from 5.5% with 0.94 M salt to 0.9% with 1.01 or 1.06 M salt. The increase of reproducibility of precipitate weight and drug yield with salt concentration likely indicates more stable, stronger and larger aggregates that could be filtered more effectively. An increase in aggregate size with an increase in the distance above the cloud point temperature has also been observed for clay particles stabilized with PEO formed at higher temperature above the cloud point. The increase in the flocculation efficiency was attributed to an increase in the tendency for the PEO to phase separate and adsorb onto the clay particles.

As the poloxamer 407 concentration was lowered 2 fold in system C relative to system B for a given salinity, the potency increased from 36.7 to 54.1%, yet the dissolution rate remained extremely high. The potency was over 2.5 times that of the overall potency in the suspension. The drug yield increased to 97.6% while the surfactant yield decreased significantly. Furthermore, the particle size changed very little. Given these beneficial results, the PVP K-15 concentration was reduced by a factor 3 in system D relative to system C and the drug yield increased to nearly 100%. The concentrations of drug and surfactant were similar, while the salt concentration decreased to 6.4%.

The selectivity for drug particles over surfactants (g precipitate drug/g filtrate drug)/(g precipitate drug/g filtrate surfactant) in the filtration process was very high and usually quite reproducible as shown is the last column of Table 4. The high deviation for system C was due to high drug yield, and thus the very small amount of drug in the filtrate. The excellent reproducibility in nearly all of the properties in Table 4 for most systems makes the flocculation/filtration concept interesting for practical application.

Based on dissolution rate and drug recovery, systems B and C flocculated at 1.01 M salt concentration may be expected to produce the highest drug bioavailability. The recommended daily dose of naproxen in adults for rheumatoid arthritis, osteoarthritis, and ankylosing spondylitis is 500-1000 mg. According to the ratio of sodium sulfate to naproxen in systems B or system C, the daily dose of sodium sulfate from these powders would be 81-286 mg. This amount is much less than the commercial daily dose from OCL®, a saline laxative (Abbott), in which 1.29 g sodium sulfate was used. Thus flocculation of naproxen nanoparticles with sodium sulfate would be worthy of consideration in the pharmaceutical industry based on lack of toxicity at the levels employed in this demonstration.

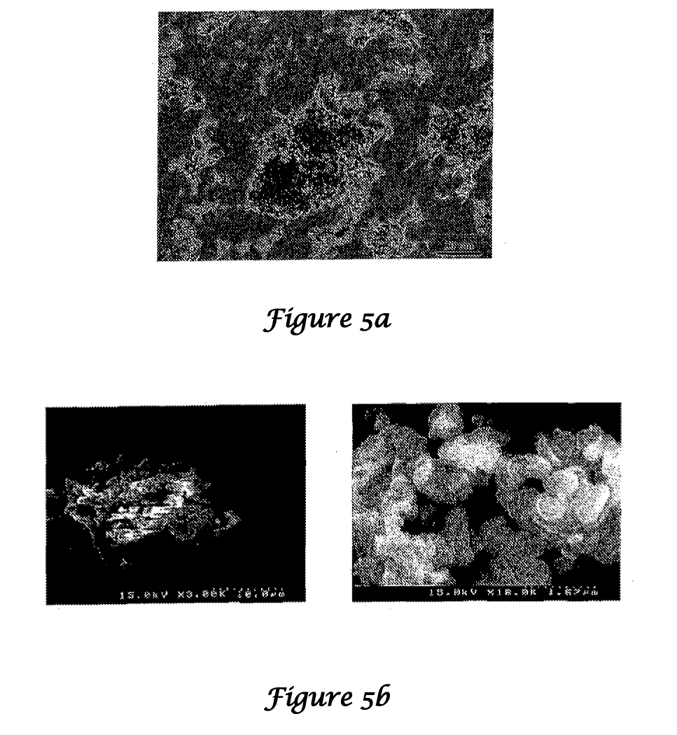

Morphology of flocculated naproxen nanoparticles is demonstrated by optical microscope and SEM. The morphology of naproxen nanoparticle flocs of system B flocculated at 1.01 M salt concentration in the suspension and after drying is shown in FIGS. 5 (a) and (b). As shown in FIG. 5 (a), naproxen flocs with sizes ranging from 5 to 150 μm were formed in the suspension. These flocs were larger than the 2 μm limit for P2 filter paper and thus more than 90% of the naproxen was recovered. As shown in FIG. 5 (b) (left panel), a floc with size larger than 30 μm was formed during flocculation and drying. This particle, however, was composed of primary nanoparticles of approximately 300 nm, shown on the right, consistent with the size measured by light scattering.

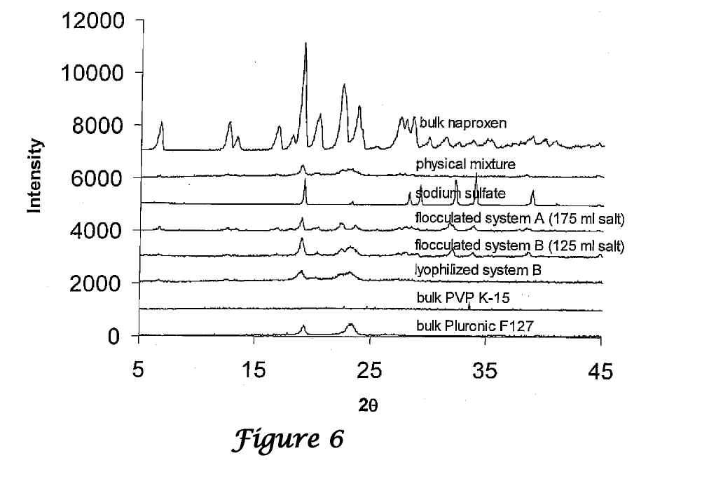

X-ray diffraction was used to analyze the crystallinity of the dry powders. As shown in FIG. 6, the largest naproxen peaks coincided with those of bulk poloxamer 407. The naproxen peaks were much smaller and broader in the physical mixtures of the various formulations as well in the dried powders produced by antisolvent precipitation. However, the naproxen peaks at 2θ=22.6° and 23.9°, especially in the high potency samples produced by flocculation, were evident. Thus, crystalline naproxen was formed whether the suspension was dried by lyophilization or flocculation followed by filtration and vacuum drying. The presence of sodium sulfate in the final powder is also apparent in the diffraction pattern.

Crystalline naproxen powders composed of submicron primary particles produced by antisolvent precipitation were successfully recovered from aqueous suspensions by flocculation with sodium sulfate followed by filtration and vacuum drying. The flocculation was reversible as the particle size upon redispersion in water under various conditions was comparable to the original particle size in the aqueous suspension prior to flocculation. Less salt was need for flocculation when the stabilizer was poloxamer 407, where the hydrophilic group was ethylene oxide, than for PVP K-15, where it was N—C═O units on lactam rings, consistent with the trends in the cloud point phase equilibria curves. The dissolution rate of dried, flocculated naproxen particles coated with stabilizers was very high when the size of the nanoparticles upon redispersion was on the order of only 300 nm. The size measured by light scattering was consistent with the primary particle size in the powders measured by SEM. The redispersibility of the dried powders was successful for a minimum salt concentration in the powder and the proper composition and concentration of the stabilizer. The dissolution rate was linearly correlated with the specific surface area calculated from the average particle diameter after redispersion. Extremely rapid dissolution, up to 95% of the powder in two minutes, was achieved for 300 nm particles. The salt concentration could be optimized to control the flocculation to balance the drug yield versus potency with excellent reproducibilities of both properties (average deviation ranged in 1-2%). The drug potencies of flocculated samples were enhanced in the filtration step by up to 61% relative to the initial value, due to removal of surfactant with the filtrate. The yield of the drug in the powder was typically 92 to 99% and the drug potency varied by only 1 to 2%. The residual sodium sulfate concentration in the dried powders, typically 10% or less was far below toxic limits based on the recommended dose of naproxen. The flocculation/filtration process simplifies drying to obtain powder relative to lyophilization. It also produces smaller particles upon redispersion of the powder than would be expected in spray drying, where the particle sizes are usually above one micron. Flocculation followed by filtration and drying is an efficient and highly reproducible process for the rapid recovery of drug nanoparticles to produce wettable powders with high drug potency and high dissolution rates.

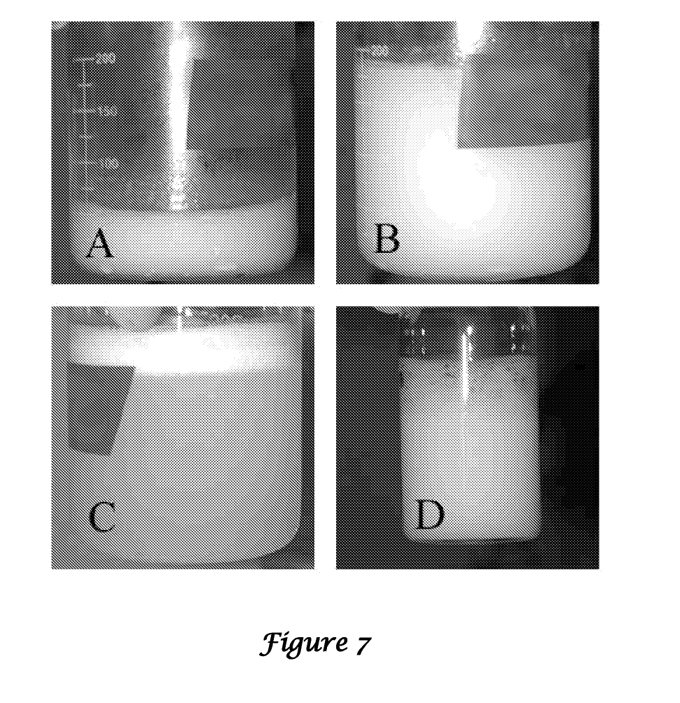

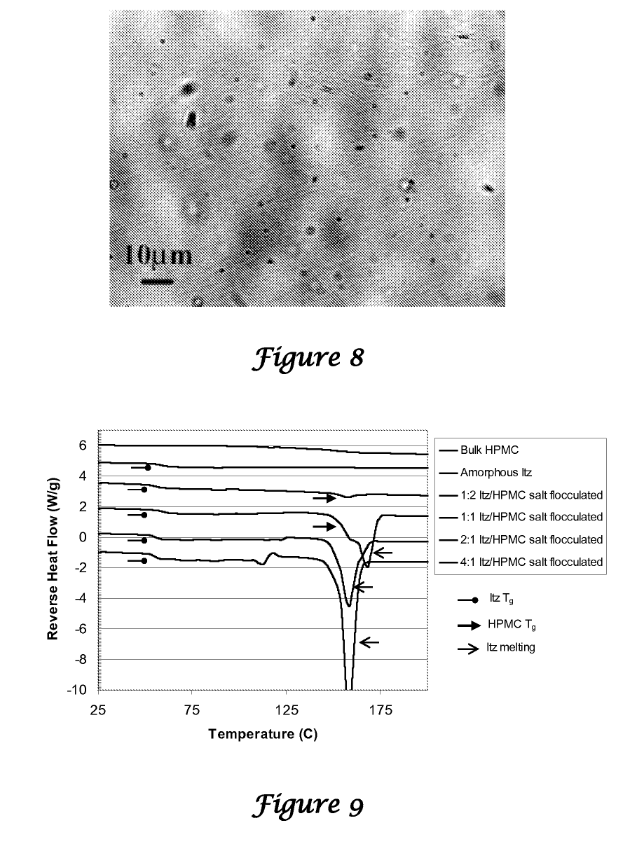

Polymer-salt cloud points, particle properties, and recovery after flocculation. The cloud point temperatures of P407 and HPMC over a range of Na₂SO₄ solutions have been reported. On the basis of extrapolation down to 25° C., molarities of approximately 0.55 and 0.8 are needed to precipitate a 2% solution of pure P407 and HPMC, respectively. For the present invention, concentrations of HPMC ranged from 0.25 to 1% (wt./vol.) (formulations A-C, Table 5) and P407 concentrations from 0.03 to 0.87% (formulations D-I, Table 6). As shown in Table 5, a minimum molarity of 0.25M (after addition of salt solution) was required at 25° C. to flocculate particles stabilized by HPMC. In order to ensure complete and rapid flocculation of nanoparticles, a molarity of 1M in the final mixture was used for all Itz/P407/HPMC samples. The excess salinity (above the minimum required, 0.25M) ensured strong attractive forces between the polymer chains. In both cases the flocculation was essentially instantaneous, in less than 1 second after mixing the salt solutions (FIG. 7-B), indicating loss of the steric stabilization by the PEO chains. Average floc sizes were about 5-7 μm, according to microscopic observation just after mixing with salt solution, shown in FIG. 8. FIG. 7-B shows the large flocculated particles took up the entire dispersion volume, and creaming of the flocs reached steady state in 3 minutes (FIG. 7-C). Spontaneous redispersion occurred upon regaining good solvent quality by adding the dried powders to pure water with gentle stirring (FIG. 7D).

TABLE 5

| | | | Drug lost | | | | | |
|---|---|---|---|---|---|---|---|---|
| | Na₂SO₄ | Filtration | in filtrate | Drug | Drug yield | Salt | Polymer | |
| | Molarity | rate | (drug wt./ | loading | (% drug | (% salt | (% pol. | |
| | to | (mL/cm² | tot. drug | (% drug wt./ | wt./tot. | wt./tot. | wt./tot. | |
| Sample | flocculate | *min) | wt. %) | tot. wt) | drug wt.) | wt.) | wt.) | Selectivity |
| A - 4:1 | 0.25 | 0.101 | 1.1 | 92 ± 0.3 | 95.4 | 0.63 | 7.4 | 200 ± 17 |
| Itz/HPMC | | | | | | | | |
| B - 2:1 | 0.25 | 0.089 | 2.9 | 90 ± 0.1 | 89.0 | 0.27 | 9.7 | 130 ± 23 |
| Itz/HPMC | | | | | | | | |
| C - 1:1 | 0.3 | 0.093 | 0.30 | 94 ± 0.7 | 101 | 0.81 | 5.2 | 6300 ± 1000 |
| Itz/HPMC | | | | | | | | |

TABLE 6

| | Theoretical | Drug loading after | | Size after | | |
|---|---|---|---|---|---|---|
| | drug loading | flocculation | Size before | redispersion and 5 | | |
| Itz/P407/ | (% drug wt./ | (% drug wt./ | flocculation | minutes sonication | | |
| HPMC ratio | tot. wt.) | tot. wt.) | (D10/D50/D90) μm | (D10/D50/D90) μm | Average θ | Supersat.AUC_{2 hr} |
| D - 32:1:8 | 78 | 87 ± 1.2 | 0.12/1.27/3.04 | 0.16/0.79/2.72 | 19 ± 3.7 | 768 |
| salt | | | | | | |
| flocculated | | | | | | |
| E - 8:1:2 | 73 | 94 | 0.11/0.34/2.26 | 0.12/0.37/1.48 | 23 ± 6.8 | 663 |
| salt | | | | | | |
| flocculated | | | | | | |
| F - 4:1:1 | 67 | 80 ± 3.6 | 0.11/0.29/1.08 | 0.12/0.33/1.09 | 38 ± 2.5 | 548 |
| Itz/P407/ | | | | | | |
| HPMC salt | | | | | | |
| flocculated | | | | | | |
| G - 8:3:2 | 61 | 88 | 0.11/0.29/2.28 | 0.11/0.31/0.97 | 33 ± 3.2 | 404 |
| Itz/P407/ | | | | | | |
| HPMC salt | | | | | | |
| flocculated | | | | | | |
| H - 8:1:2 | 73 | 90 ± 2.0 | 0.20/0.68/1.65 | 0.21/0.81/1.81 | 31 ± 6.8 | NA |
| Itz/P407/ | | | | | | |
| HPMC | | | | | | |
| homogenized | | | | | | |
| & salt | | | | | | |
| flocculated | | | | | | |
| I - 8:1:2 | 73 | NA | 0.11/0.28/0.80 | 0.14/0.70/5.80 | 51 ± 3.3 | NA |
| Itz/P407/ | | | | | | |
| HPMC rapidly | | | | | | |
| frozen, not | | | | | | |
| salt flocculated | | | | | | |
| J - 8:1:2 | 73 | NA | 0.11/0.34/2.26 | NA | NA | 1120 |
| Itz/P407/ | | | | | | |
| HPMC | | | | | | |
| suspension, | | | | | | |
| no drying | | | | | | |
| K - 8:1:2 | 73 | NA | 0.11/0.34/2.26 | 0.27/5.18/10.8 | NA | NA |
| Itz/P407/ | | | | | | |
| HPMC | | | | | | |
| spray dried | | | | | | |
| L - 8:1:2 | 73 | NA | 0.11/0.34/2.26 | 2.30/5.96/40.1 | NA | NA |
| Itz/P407/ | | | | | | |
| HPMC | | | | | | |
| dispersion, | | | | | | |
| slowly heated | | | | | | |
| M - 8:1:2 | 73 | NA | 0.12/0.19/0.34* | 0.17/0.23/0.31* | NA | NA |
| Itz/P407/ | | | | | | |
| HPMC | | | | | | |
| dispersion, | | | | | | |
| rapidly heated | | | | | | |

The sodium sulfate concentration in the filtrate after rinsing with 30 ml of the HPMC solution was verified using conductivity and in all cases, the weight of salt in the filtrate was more than 90% of the total salt added. The final powders from the filter cake contained less than 1% Na₂SO₄ by weight. HPLC analysis of the filtrate showed no more than 3% (drug wt. in filtrate/tot. wt. drug) of the drug was lost through the filter, indicating yields higher than 97%. The residual salt in the final dried powder corresponded to less than 2.5 mg per 200 mg dose of Itz (based on 90% drug loading). This quantity is well below the 120 mg of Na₂SO₄ allowed in an oral dosage form, according to the inactive ingredient guide published by the FDA.

Filtration rates of the Itz dispersions, reported in Table 5, were similar to those of flocculated naproxen nanoparticle suspensions in a previous demonstration. In all formulations, free polymer, which was not bound to drug particles, was removed with the filtrate, producing drug loadings typically around 90% (drug wt./tot. wt.), as tabulated in Tables 5 and 6. Polymer loading was determined by a simple mass balance and reported as % polymer wt./tot. wt. Extremely high filtration selectivities for drug particles over polymer, (g drug precipitate/g drug filtrate)/(g polymer precipitate/g polymer filtrate) were observed, as reported in Table 5, and were similar to values reported by the flocculation of naproxen.

Particle Morphology after Flocculation and Filtration: Using light scattering, particle sizes were measured after removal of organic solvent from the precipitated dispersions and after redispersion of dried powders into water. Without the use of P407 as a stabilizer, particle sizes of aqueous dispersions just after organic solvent removal were large (1-5 μm diameters), despite dried powder surface areas as high as 51 m²/g. Addition of P407 to the formulation mitigated agglomeration of nanoparticles during antisolvent precipitation and light scattering measurements, prior to addition of salt, gave sizes in the submicron range. Increasing the P407 concentration led to a decrease in average particle size until a threshold was reached at approximately 300 nm, shown in Table 6. This trend has been observed in previous works where the ratio of the mixing time and precipitation time, the dimensionless Damkohler number, was decreased by either increasing stabilizer concentration or mixing energy. The particle size decreased until a plateau was reached where the Damkohler number was <1.

As listed in Table 6, the average sizes of P407/HPMC systems after redispersion were typically within 40 nm of the original dispersion before flocculation. For rapidly frozen and lyophilized 8:1:2 ITZ/P407/HPMC, the average size after redispersion was 700 nm, over twice the original value, see Table 6. The spray dried 8:1:2 ITZ/P407/HPMC average size was quite large, 5 μm, after redispersion. Contact angles (reported Table 6) of all salt flocculated samples, including the homogenized control, were all approximately 20-40° regardless of the formulation. The rapidly frozen and lyophilized control had a much higher angle of 51°. Pure ITZ and HPMC had contact angles of 79° and 66°, respectively.

Modulated DSC of formulations A-C revealed the presence of two glass transitions, one for ITZ (about 58° C.) and HPMC (about 154° C.) as seen in FIG. 9. These two T_{g}s indicated two distinct phases in the drug nanoparticles. Previous work with precipitated ITZ/HPMC systems established a core-shell arrangement of drug and polymer, as evident from surface content analysis via X-ray photoelectron microscopy and contact angle measurements. As the polymer is water soluble, its adsorption to the surface of precipitated hydrophobic surfaces orients the polymer primarily to the surface of the particles, lowering the interfacial energy of the system. This surface orientation facilitates the use of very little polymer to stabilize nanoparticles with high drug loading.

In addition to hydrophilic polymers, excess salt was detected on the surface of dried particles by contact angle measurements with pH 6.8 buffer. Contact angles of salt flocculated powders, including the homogenized sample, were much lower than for both pure ITZ (79°±13) and HPMC (66°±3.9) alone. For the lyophilized control, it was lower than that of pure HPMC from the presence of P407 on the surface, but still higher than those of the flocculated samples. The more favorable wetting for the flocculated particles has the potential to accelerate dissolution.

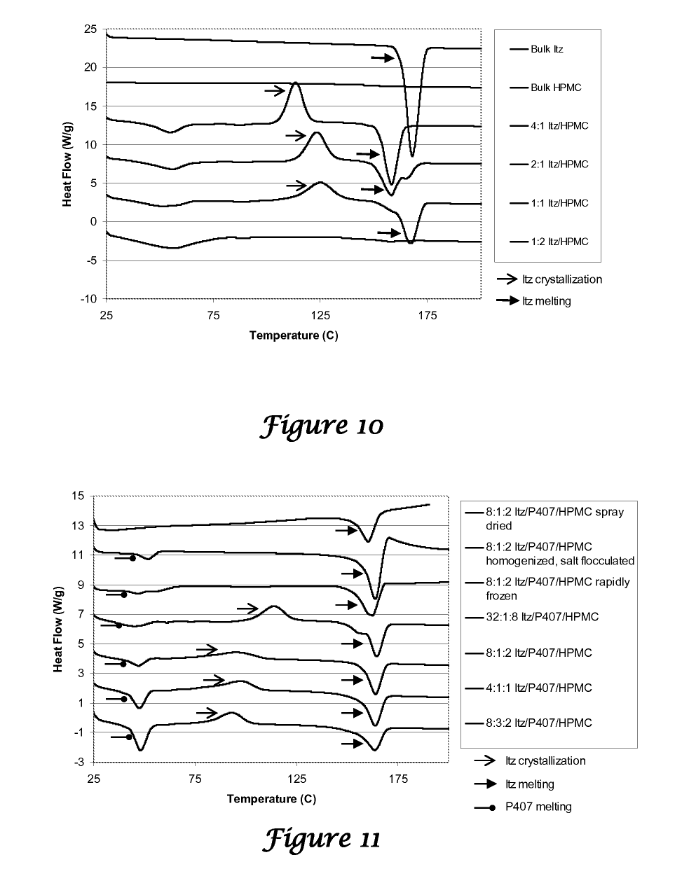

The crystallization of amorphous drug during mDSC heating was evident in an exothermic peak ranging from 90-130°, as shown in FIGS. 10 and 11. The crystallization temperature increased with HPMC content, FIG. 10, and decreased with P407 content, FIG. 11. The change in kinetics of crystallization may be explained by a decrease in mobility with an increase in HPMC and an increase with P407. Melting peaks of the in-situ crystallized drug are observed in both FIGS. 10 and 11, at about 168° C. In FIG. 9, the melting peak of ITZ overlapped with the T_{g} peak for HPMC. In FIG. 11, the melting peak for P407 can also be seen at about 45° C., which masked the T_{g} of ITZ. In all cases the presence of a glass transition for ITZ and/or a crystallization peak verified the amorphous character of flocculated, dried powders. Homogenized, rapidly frozen, and spray dried 8:1:2 ITZ/P407/HPMC controls, (Table 6; formulations H, I, and K, respectively), exhibited no evidence of amorphous drug as there was no crystallization peak, but a large melting peak at about 168° C.

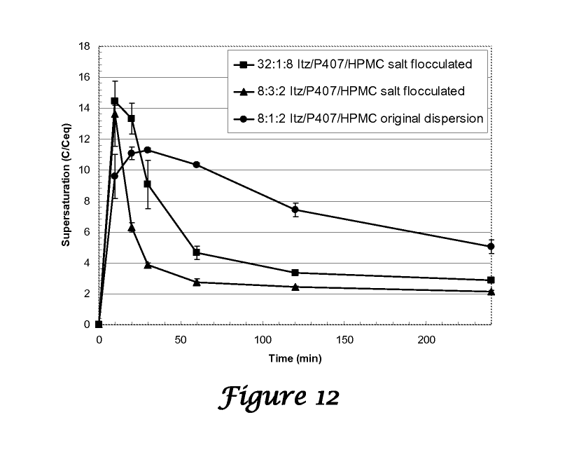

The supersaturation curves and the calculated areas under the curve (AUC) are shown in FIG. 12 and Table 6, respectively. In the case of dried powders, an amount equal to 25-times the equilibrium solubility was added at time zero. For the 8:1:2 ITZ/P407/HPMC original nanoparticle dispersion, drops were added slowly to minimize excess particles, which would otherwise facilitate heterogeneous nucleation. It has been shown recently that the supersaturation produced by this technique may approach the metastable solubility limit in pH 6.8 buffer with 0.17% SDS. The maximum in the supersaturation level for salt flocculated, dried powders was about 14, even greater than that for the original nanoparticle dispersion (shown in FIG. 12). Dissolution rates were rapid for all supersaturation curves, as expected from high surface areas and a large driving force from the high metastable solubility. For flocculated samples, however, the supersaturation decayed more rapidly than for the 8:1:2 ITZ/P407/HPMC dispersion. The supersaturation can be reduced by incorporation of the dissolved ITZ into the excess particles. The slower decay in supersaturation, and consequently, larger AUC for the original dispersion is likely caused by the lack of excess solid particles. Similarly, the larger (lower surface area/volume) excess particles in the 32:1:8 flocculated sample may explain its larger AUC relative to the other three flocculated samples.

The lyophilized samples crystallized as a result of high mobility during water sublimation caused by the low T_{g} P407. Since crystallization of drug occurred in both the rapidly frozen/lyophilized and spray dried 8:1:2 Itz/P407/HPMC controls, as evident by mDSC in FIG. 11, dissolution demonstrations were not performed. Thus the lack of crystallization in the salt flocculation process is a significant advantage.

Flocculation of sterically stabilized dispersions by salt: The lowering of the cloud point temperature of PEO or HPMC with electrolytes has received significant attention. Structural incompatibility of the hydrophilic hydration layer about the ether oxygen atoms of PEO with anion-associated water molecules leads to desolvation of the polymer chains. As the polymer chains are desolvated, the segmental interactions of polymer chains become attractive and the chains collapse. For colloids with adsorbed PEO, the interparticle attractive forces of the desolvated polymer layer cause flocculation of the particles.

For rapid diffusion limited Smoluchowski flocculation, in the limit of no stabilization, each collision is sticky and the rate of change in number of particles is given by:

rₛ=kₛnₚ² (1)

where nₚ is number of particles per volume (mL⁻¹) and kₛ is calculated from the temperature and kinematic viscosity of the medium. Under very poor solvent conditions, flocculation for PEO stabilized particles is rapid, and the stability ratio, that is, rₛ/actual rate, is close to 1. For PEO stabilized colloids, the solvent quality has been varied by raising temperature to achieve stability ratios of 2.3.

The ratio of particle diameter to stabilizing polymer layer, a/δ was used to delineate the importance of van der Waals core-core versus polymer-polymer interactions. In the demonstrations, particles sizes of about 300 nm with an adsorbed PEO layer of about 15 nm in length have relatively thick polymer layers, where a/δ=20. For poor solvent conditions, the collapsed polymer layers produce strong attractive forces between the particles, whereas the core-core attraction is minor Excess salt was added to the dispersions, above the minimum critical flocculation concentration to produce strong attractive forces and rapid flocculation seen in FIG. 7. For a stability ratio approaching unity, Equation 1 predicts the number density of particles will be reduced by half after only 0.11 sec. for nₚ was about 1.4×10¹² mL⁻¹, in good agreement with the instantaneous flocculation observed visually. The flocculation rates could not be measured directly with light scattering; however, since the flocculation was so rapid and the dispersions were extremely turbid.

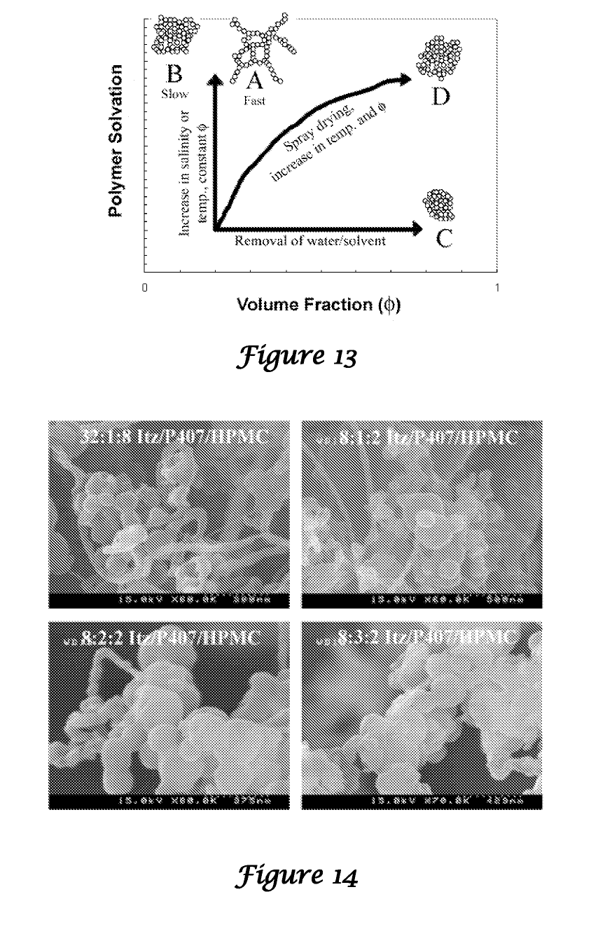

The floc structure depends on both the interparticle forces and the volume fraction of particles, φ as illustrated by the schematic in FIG. 13 Immediately after the addition of excess salt, φ in pathway A is essentially constant, prior to creaming, as the volume of the aqueous dispersion remains constant. Here, the rapid generation of strong interparticle attractive forces forms an interconnected network with a loose, open fractal structure (pathway A in FIG. 13). As shown in Table 6 and the SEMs of FIG. 14, the particles redispersed to their original size in good solvent conditions, behavior indicative of loose flocs. The very small 300 nm primary particles diffuse rapidly and bind to a primary particle already on the growing floc. The strong attraction inhibits significant rearrangement of the loose floc to minimize surface area, preserving an open floc.

The fractal dimension of a floc, D_{f}, characterizes the floc structure by relating the volume fraction of solid in the floc, φₖ to the primary particle diameter, d, and the floc diameter, dₖ.

$${φ}_{k} = {( \frac{{d}_{k}}{d} )}^{{D}_{f} - 3} \qquad ( 2 )$$

For a floc composed of densely packed particles, D_{f} approaches 3. For more open structures, or fractals produced by rapid diffusion-limited aggregation, D_{f} is typically about 1.7. As shown in FIG. 7-C, 3 minutes after addition of salt solution, the flocs creamed to a steady state volume. Assuming the flocs are at the closest packed density in this cream layer, φₖ can be assumed to be approximately equal to φ in the cream layer. The cream layer volume was approximately half the volume of the original dispersion, shown in FIG. 7-A, indicating φ increased from 0.01 to 0.02. Based on the measured values of d=300 nm and dₖ=7 μm, D_{f} is approximately 1.76 for salt flocculated Itz/P407/HPMC dispersions, representative of an open floc. With D_{f}<<3, the flocs are loose and open in structure consistent with the essentially complete redispersion once good solvent conditions are reestablished, as shown in Table 6.

Upon returning the thick (a/δ=20) stabilizing polymer to good solvent conditions, the weak attractive forces between particle cores is easily overcome by the solvation of the polymer chains. In the flocculated state, hydrogen bonding between PEO chains is relatively weak, due to only one terminal hydrogen bond donor group per chain. With the low melting point of PEO, reflecting weak intermolecular forces, the collapsed chains are non-crystalline, and are easily solvated upon redispersion in water. The primary particles in the open flocs are highly accessible to the solvent with high surface area. Also, the residual salt in the final powder was sufficiently low to allow full solvation of stabilizing moieties. Thus, the solvation of the thick polymer layers on the accessible particles in the open flocs with low residual salt leads to essentially complete redispersion to the original particle size of 300 nm.

During mixing of salt solution with the nanoparticle dispersion, the overall volume of the system increases, decreasing φ from about 0.01 to about 0.003, as the flocs take up the entire dispersion volume (FIG. 7-B) Immediately after mixing, the flocculation takes place under very dilute and constant φ conditions, as shown by pathway A in FIG. 13, to form loose open flocs. Filtration removes water and solvent in a separate step after the floc is formed. In contrast, φ increases continually in freezing processes as the water and/or organic solvents are frozen (pathway C, FIG. 13). φₖ must be equal to or greater than φ, therefore the removal of water and/or solvent increases the minimum possible floc density. The maximum value of φ is 0.74, assuming close packed spheres. Based on equation 2, with d=280 nm and dₖ=700 nm (Table 2) from rapidly frozen 8:1:2 ITZ/P407/HPMC, an increase in δₖ from 0.01 to 0.74 gives an increase in D_{f} from 2.4 to 2.96 (reported in Table 7). The high fractal dimension suggests a much more densely packed floc formed by rapid freezing than salt flocculation, consistent with much larger particle sizes upon redispersion of the former.

Slowly heating the 8:1:2 ITZ/P407/HPMC nanoparticle dispersion to 92° C., at relatively constant φ with limited evaporation (pathway B, FIG. 13), resulted in slow, irreversible flocculation, as the particles redispersed at ambient temperature to a large size of about 5 μm even with sonication (Table 6). Since 92° C. is just below the cloud point of PEO in water (about 100° C.), flocculation was slow from weak attractive forces of only partially collapsed polymer layers. In contrast, dispersions of 300 nm 8:1:2 ITZ/P407/HPMC particles were heated rapidly just to the cloud point temperature at 98° C., under constant φ conditions (pathway A, FIG. 13). Rapid heating without evaporation formed open flocs, which redispersed to their original size upon cooling (Table 6). Bevan and Prieve also showed reversible flocculation from rapid heating of PEO-stabilized polystyrene latex spheres. Thus, the rate of change in PEO solvency directly impacts the structure of the floc, as indicated by its size upon redispersion in water.

During spray drying, the evaporation of water causes a marked increase in φ, simultaneously with an increase in temperature, as shown by pathway D in FIG. 13. Since the dispersion is heated to 140° C., much higher than the cloud point of PEO, the polymer becomes desolvated. Heating of the about 20 μm high surface area droplets occurs in msececonds, as evident by the fact that droplets evaporate before hitting the side walls of the spray dryer. The rapid rate of heating and thus, desolvation of the thick stabilizer shell, produces strong attractive forces between the particles. However, with the simultaneous increase in φ, the flocs become densely packed, and may be further compressed by the large capillary forces of the shrinking droplets. Thus, the aggregates do not redisperse to the primary particle size, as reported in Table 6. The final particle size can be estimated by assuming all particles present in the initial droplet create a single larger particle upon water evaporation.

Under the conditions of spray drying as detailed above, droplets are 10-30 μm, based on measurements made by Engstrom. With an initial particle concentration of 10 mg/mL and a particle density of 1.3 g/cm³ (based on the bulk drug density), a single particle of 4.4 μm would be produced from each 20 μm drop, assuming closest packed spheres. This value is very close to the demonstrated particle size of 5 μm upon redispersion. As reported in Table 7, when φ is assumed to be 0.74, the closest packed limit, the fractal dimension of the flocs is approximately 2.90, indicative of densely packed flocs. Therefore, the ability to maintain a low nearly constant value of φ in the salt flocculation process is highly advantageous for producing open flocs which are highly redispersible, unlike the denser flocs produced by the freezing and spray drying processes where φ increased markedly.

TABLE 7

| Salt flocculated | | Rapidly frozen | | Spray dried | |
|---|---|---|---|---|---|
| dₖ/d = 7 μm/300 nm | | dₖ/d = 700 nm/280 nm | | dₖ/d = 5 μm/300 nm | |
| φₖ | D_{f} | φₖ | D_{f} | φₖ | D_{f} |
| 0.003 | 1.2 | 0.01 | 2.4 | 0.01 | 1.4 |
| 0.02 | 1.8 | 0.67 | 3.0 | 0.67 | 2.9 |

Increase in drug loading by flocculation and filtration. In addition to forming a redispersible floc, salt flocculation and filtration also offers the advantage of removing excess polymer from the final powder, as is evident from the increase in drug loadings reported in Tables 5 and 6. As the large, flocculated particles are trapped on top of the filter, free unadsorbed polymer escapes with the filtrate. Although the free polymer is also desolvated by salt and forms a collapsed structure, its effective diameter is on the order of 10 nm, based on measured PEO coil lengths under poor solvent conditions. Since the concentrations of polymer in solution are low, approximately 1.67 mg/mL (P407) and 2.5 mg/mL (HPMC), the collapsed free polymers were not likely to flocculate to high enough diameters to be trapped by the filter paper with 1-5 μm pores. Additionally, in a control demonstration, pure polymer solutions of 1.67 mg/mL P407 and 2.5 mg/mL HPMC were flocculated with 1M Na₂SO₄ and passed through the filter to produce a cloudy filtrate with minimal polymer on the filter paper. For polymer stabilized dispersions, the high selectivity for particles versus free polymer during filtration is evident from the large values (150-6300) reported in Table 5. During solution precipitation, as well as milling processes, to form nanoparticles a high concentration of polymer in solution is highly beneficial for generating a large driving force for adsorption to particle surfaces to inhibit particle growth. Once the particles are formed and passivated with the stabilizer, the results indicate that the unbound polymer is no longer needed and can be removed without further particle growth.

Preservation of amorphous drugs by flocculation and filtration: Flocculation and filtration were performed at low temperatures to minimize particle growth and crystallization of the drug. As observed in mDSC thermograms, temperatures as low as 90° C. leads to the crystallization of amorphous ITZ. Spray drying, which requires temperatures in this range, caused crystallization of the amorphous precipitated particles, as shown by the single melting peak of ITZ in FIG. 11. For salt flocculation, the rapid filtration at low temperatures mitigated crystallization as was evident in mDSC and supersaturation demonstratons. Spray drying forces particles close together as the water is removed by evaporation or ice formation and the concentration of free excipients also increases. Both of these factors enhance the rate of particle growth and crystallization.

When one or both of the polymers exhibits a low T_{g}, such as P407, removal of the excess polymer with filtration can reduce crystallization tendency of the drug. As shown in the mDSC thermograms in FIG. 11, the crystallization temperature of amorphous ITZ decreased with increasing P407 content. P407 melts around 50° C., where Itz can dissolve in the molten polymer, resulting in solvent-mediated crystallization of drug. In contrast, the higher T_{g} polymer, HPMC, increased the crystallization temperature, as apparent in the mDSC curves in FIG. 10. According to the Gordon Taylor equation, the fraction of high T_{g} material may be determined to achieve a reasonable composite T_{g}. Also, removal of excess polymer (including low T_{g} material) in the final powder by filtration is beneficial for the storage stability of amorphous nanoparticles. At low temperatures, rapid removal of solvent and excess low T_{g} polymer immediately after precipitation, flocculation, and filtration, helps maintain an amorphous state, in contrast to spray drying where high temperatures and concentration of the particles and free excipients caused crystallization.

Dissolution of flocculated particles to produce supersaturated solutions: The rate of dissolution has been shown to influence the maximum level of supersaturation. The undissolved solid is susceptible to solvent-mediated crystallization during the dissolution process, upon contact with water. In previous work, amorphous pure ITZ particles were dissolved in acidic media and the maximum supersaturation levels were correlated to the surface area available for dissolution. Rapidly dissolving amorphous nanoparticles have less time to crystallize in the presence of dissolution media. In another demonstration, pre-wet ITZ particles in suspension created the highest supersaturation levels, which were close to the theoretical solubility predicted by a heat capacity corrected Gibbs free energy difference between the amorphous and crystalline drug. Likewise, the rapidly dissolving salt flocculated powders produced supersaturation levels approaching the metastable solubility limit, as estimated by dropwise addition of the nanoparticle dispersion (without salt flocculation) to pH 6.8 media (FIG. 12). In contrast, dissolution of low surface area amorphous ITZ made by solvent evaporation with HPMC only generated supersaturation levels up to 2.5 in pH 6.8 media. Therefore, the high surface area, polymer coated, amorphous particles formed by the salt flocculation process produce much higher supersaturation levels than low surface area solid dispersions.

The simple flocculation/filtration process may be used to recover nanoparticles from aqueous dispersions rapidly and efficiently, with yields on the order of 90%, while maintaining amorphous primary particles that may be dissolved in aqueous media to achieve high supersaturation levels. Addition of salt to polymer-stabilized amorphous nanoparticle dispersions collapsed the stabilizer chains to produce large flocs, which were filtered rapidly. Large amounts of stabilizers may be used in the particle formation stage to minimize particle growth, and then excess stabilizer may be removed in the filtration step to achieve high drug loadings up to 90%. The rapid change in interparticle forces, under constant particle volume fraction (φ), produced open flocs (low fractal dimension) and excellent redispersion in water to the original particle size of 300 nm. In contrast, drying of nanoparticle dispersions by either spray drying or rapid freezing/lyophilization produced densely packed flocs (high fractal dimension), which did not redisperse to form submicron particles.

For the salt flocculation/filtration process, the low temperature and constant dilute conditions, as well as rapid removal of solvent, inhibited both growth and crystallization of the amorphous primary nanoparticles. High supersaturation levels up to 14-times the crystalline solubility in pH 6.8 media were facilitated by rapid dissolution of the nanoparticles prior to crystallization in the presence of the media. High supersaturation levels would have the potential to improve absorption markedly of poorly water-soluble drugs in the gastrointestinal tract. However, for particles produced by spray drying, the increase in φ and high temperature led to crystallization of the nanoparticles and with yields well below the values of 90% in the flocculation/filtration process. The ability to recover nanoparticles from aqueous dispersions rapidly by forming loose flocs, which are readily redispersible to the original particle size, and without the need to evaporate the water, is of general interest for all types of nanoparticles including pharmaceuticals.

### Example 1

A 5% naproxen and 2% Pluronic F127 solution in ethanol was pumped through a stainless tube with i.d. of 1/16 in. into a graduated cylinder containing 50 ml aqueous solution. The stabilizer used in aqueous solution was 3% PVP K-15. The flow rate was 1 ml/min. The organic solution was pumped for 5 minutes with constant magnetic stirring. The final suspension concentration of naproxen was 5 mg/ml water. Three demonstrations were done at the same conditions to check the reproducibility of the process. After the spray, the particle size of the suspension was measured with Malvem Mastersizer-S. The large particles were filterable. 45 ml of the suspension was filtered with a 0.45 μm nylon membrane filter (WHATMAN®) under a vacuum aspirator. The diameter of the filter was 47 mm. The particles collected on the top of the membrane were dried in a vacuum oven under room temperature and −29 in. Hg. Half of the dried particles were redispersed into 15 ml of pure water and other half were redispersed into 15 ml of 1% PVP K-15. The redispersions were sonicated for 5 minutes with a Branson sonifier 250 (VWR). The particle size was then measured with Malvern Mastersizer again. The average particle size was less than 400 nm indicating that the large aggregates that were filter could be broken by sonication after redispersion. The operating temperature and particle sizes of naproxen before and after filtration are listed in Table 8. See Table 8 below:

| | | | | Particle size of | |
|---|---|---|---|---|---|
| | | | | redispersion in | Particle size of |
| | | Particle size of the | | water after | redispersion in 1% |
| | | original suspension | Particle size of | filtration, drying | PVP K-15 after |
| | | before sonication | original suspension | and sonication | filtration, drying and |
| | T | and filtration (μm) | after sonication (μm) | (μm) | sonication (μm) |
| Exp # | (° C.) | D_{0.1}/D_{0.5}/D_{0.9} | D_{0.1}/D_{0.5}/D_{0.9} | D_{0.1}/D_{0.5}/D_{0.9} | D_{0.1}/D_{0.5}/D_{0.9} |
| 1 | 4.7 | 32.71/145.12/336.29 | 0.10/0.32/5.42 | 0.12/0.34/2.23 | 0.17/0.39/2.43 |
| 2 | 3.9 | 0.41/59.31/184.52 | 0.10/0.30/3.25 | 0.13/0.34/1.36 | 0.1/0.29/2.16 |
| 3 | 4.4 | 8.18/93.07/257.79 | 0.10/0.30/3.90 | 0.15/0.35/1.55 | 0.15/0.37/2.52 |

### Example 2

A solution containing 2.5% β-carotene in tetrahydrofuran as heated to 45° C. to ensure complete dissolution. The organic solution was then allowed to cool to room temperature. An aqueous solution containing PVP as a stabilizer was maintained at 5° C. and rigorously stirred with a Teflon® coated magnetic stir bar. An HPLC pump (Constametric 3200) and 1/16″ stainless steel tubing were used to introduce the organic solution into 50 ml aqueous solution at a rate of 1 ml/min. The organic solution was atomized into the aqueous solution by crimping the end of the stainless steel tubing and filing until a pressure drop around 3000 psi was obtained. After 20 minutes of spray, corresponding to a drug suspension concentration of 10 mg/ml, the particle size of β-carotene suspension was determined using a Malvern Mastersizer S. The large particles could be filtered. After the initial size was measured, the suspension was sonicated in 1 minute intervals until the meal particle size no longer decreased with additional sonication. Nanoparticle with an average diameter less than 500 nm were formed. Particles sizes for two different concentrations of PVP are reported in table 9 below. Percentages of particles at each size are based on volume.

TABLE 9

| PVP | Initial particle | Particle size |
|---|---|---|
| concentration | size (μm) | after sonication (μm) |
| (% w/v) | D_{0.1}/D_{0.5}/D_{0.9} | D_{0.1}/D_{0.5}/D_{0.9} |
| 2 | 0.19/22.72/61.55 | 0.19/0.45/16.69 |
| 3 | 0.24/22.93/42.93 | 0.11/0.30/3.53 |

### Example 3

Residual salt concentrations of salt flocculated nanoparticles. An organic solution of 3.33% itraconazole in 1,3-dioxolane was added using a 19G needle and syringe to an HPMC E5 solution in water, varying the HPMC concentration. Immediately after precipitation, 120 mL of 1.5M Na₂SO₄ was added to 50 mL of the suspension and sat unstirred for 3 minutes. The nanoparticles flocculated and were able to be filtered with fine porosity P2 type Fisherbrand filter paper (pore size about 5 μm). The filtrate was clear and contained an undetectable amount of drug, according to HPLC. The filter cake was dried at ambient conditions overnight. The filter cake was then removed from the filter and the powder was dissolved in 50:50 water/acetonitrile. Drug concentrations were determined by HPLC and salt concentrations were quantified by conductivity. The resulting drug loading and residual salt content of the dried powders are listed below in table 10. See Table 10 below:

| | Residual Salt | Drug Loading | Filtration Rate |
|---|---|---|---|
| Itz/HPMC | (wt %) | (wt %) | (mL/cm²min) |
| 4:1 Itz/HPMC | 0.63 | 92 | 0.10 |
| 2:1 Itz/HPMC | 0.27 | 90 | 0.09 |
| 1:1 Itz/HPMC | 0.81 | 94 | 0.09 |

### Example 4

Redispersion of salt flocculated itraconazole nanoparticles of varied drug loading. An organic solution of 3.33% itraconazole in 1,3-dioxolane with poloxamer 407 (F407) at various amounts was added using a 19G needle and syringe to a 2.5 mg/mL HPMC E5 solution in water. The resulting precipitated submicron particles had an average size ranging from 290 nm to 1.27 μm. Immediately after precipitation, 120 mL of 1.5M Na₂SO₄ was added to 50 mL of the suspension and sat unstirred for 3 minutes. The nanoparticles flocculated and were able to be filtered with fine porosity P2 type Fisherbrand filter paper (pore size about 5 μm). The filtrate was clear and contained an undetectable amount of drug, according to HPLC. The filter cake was dried at ambient conditions overnight. The filter cake was then removed from the filter and redispersed in pure water with sonication by a Branson Sonifier 250 at 50% duty cycle for 5 minutes. The size of the original suspension and the redispersed filter cake are listed in Table 11 below. Scanning electron microscopy, shown in FIG. 14, was taken after the dried powders were redispersed in pure water and flash frozen onto an aluminum stage, followed by lyophilization. Sizes observed by SEM were in good agreement with redispersion sizes reported by light scattering.

TABLE 11

| | | | Redispersed size |
|---|---|---|---|
| | Drug | Original size | after salt flocculation |
| | Loading | D10/D50/D90 | D10/D50/D90 |
| Drug/P407/HPMC E5 | (%) | (μm) | (μm) |
| 32:1:8 | 78.0 | 0.12/1.27/3.04 | 0.16/0.79/2.72 |
| 16:1:4 | 76.2 | 0.14/0.47/2.47 | 0.14/0.61/2.82 |
| 8:1:2 | 72.7 | 0.11/0.34/2.26 | 0.12/0.37/1.48 |
| 4:1:1 | 66.7 | 0.12/0.33/1.08 | 0.12/0.33/1.09 |
| 8:3:2 | 61.5 | 0.11/0.29/2.28 | 0.11/0.31/0.97 |
| 8:7:2 | 47.1 | 0.10/0.30/2.98 | 0.11/0.31/1.44 |

### Example 5

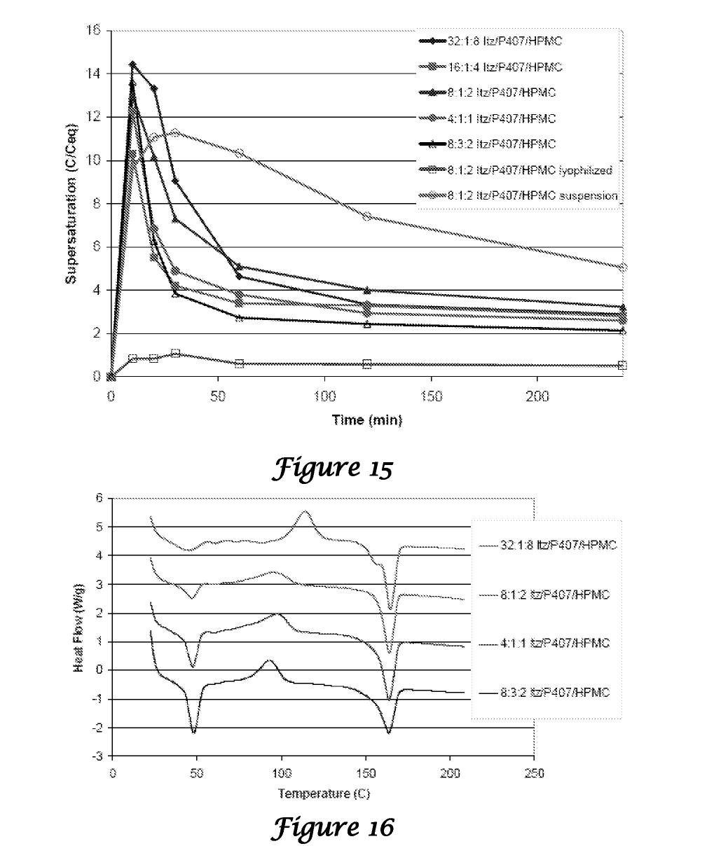

Supersaturation of basic media from salt flocculated itraconazole nanoparticles. Selected itraconazole powders produced as described in Example 3 were added to pH 6.8, sodium phosphate buffer solution with 0.17% SDS. Approximately 25-times the crystalline solubility (14 μg/mL) was added to 50 mL of dissolution media and aliquots were taken at 10, 20, 30, 60, 120, and 240 minutes. The aliquots were immediately filtered with a 200 nm filter followed by dilution by ½ with acetonitrile to prevent precipitation prior to drug concentration analysis. Drug concentrations were determined by HPLC using a Alltech 5 μm Inertsil ODS-2 C18 reverse-phase column, and detection at a wavelength of 263 nm. In the case of 8:1:2 Itz/P407/HPMC, comparison was made to a freeze dried sample as well as dissolution of the suspension prior to any particle isolation. The dissolution profiles are shown in FIG. 15, reported as supersaturation versus time. Supersaturation is defined as the drug concentration, C, divided by the crystalline solubility, 14 μg/mL in the dissolution media. Maximum supersaturations were as high as 14-times the crystalline solubility. Comparison to the lyophilized standard suggests that the release of the salt flocculated samples allows more stable supersaturation levels, which would lead to higher bioavailability for oral delivery.

### Example 6

Morphology of Salt Flocculated Itraconazole Nanoparticles. After salt flocculation and filtration, as described in Example 4, dried powders were analyzed by modulated differential scanning calorimetry, shown in FIG. 16. The amorphous content, as evident from the recrystallization peak observed at approximately 90-120° C., was preserved throughout the isolation process. Also observed were melting peaks for poloxarmer 407, at −48° C., and recrystallized itraconazole, at about 165° C.

### Example 7

Salt flocculation of wet milled itraconazole nanoparticles. Itraconazole nanoparticles were produced by high pressure homogenization by adding a ratio of 8:1:2 Itraconazole/Poloxamer 407/HPMC E5 to water followed by milling for approximately 12 hours (about 40 passes at 5000-10000 psi and −200 passes at 10000-25000 psi). The final suspension contained a particle size distribution (D10/D50/D90) of 0.20/0.68/1.65 microns, according to light scattering by Malvem Mastersizer. The nanoparticles in suspension were flocculated by adding 120 mL of 1.5M Na₂SO₄ to 50 mL of suspension. After allowing to sit for 3 minutes, the flocculated suspension was then filtered with fine porosity P2 type Fisherbrand filter paper. The filtrate was clear and contained an undetectable amount of Itraconazole, according to HPLC. The filter cake was allowed to dry overnight at ambient conditions and the dried powders were redispersed in pure water. The redispersed particle size, according to light scattering by Malvern Mastersizer was 810 nm.

The present invention related to the formation of amorphous nanoparticle aggregates by a salt flocculation and filtration process, which gives improved properties for forming and maintaining supersaturated solutions. Even though the salt flocculation process is fairly well known and reported by Chen et al., the desirable properties of the harvested particles were not anticipated.

The previous work by Chen et al. has shown that salt flocculation of crystalline nanoparticle dispersions creates dried crystalline particles that redisperse to their original size (about 300 nm diameter), according to SEM and static light scattering measurements. In certain embodiments, the current invention is the processing of amorphous rather than crystalline nanoparticles from aqueous dispersions while maintaining the amorphous morphology. Furthermore, the new salt flocculation process reduces the surface area of polymerically stabilized amorphous nanoparticles as the particle size increased by about an order of magnitude. In contrast, the particle size remained constant in the work of Chen et al.

In certain embodiments, the present invention demonstrates the supersaturation conditions obtainable of a poorly-water soluble compound in aqueous media, and how this condition can be maintained over an extended period of time. The crystalline particles studied by Chen et al. do not form supersaturated solutions. Supersaturated solutions are known to improve bioavailability of poorly water-soluble drugs.

In an embodiment, the process consists of rapidly flocculating polymerically stabilized nanoparticles by the addition of salt to or change in pH of the dispersion medium. The flocculated amorphous nanoparticle dispersion can then be rapidly filtered to remove water, additional solvents, excess unbound stabilizers and soluble salts. Rinsing the filter cake with a polymer aqueous solution minimizes the residual salt remaining to less than 1% of the total weight of the dried final particles. Previous studies by Chen et al. did not include the rinsing step.

The current invention shows the application of salt or pH flocculation to polymerically stabilized amorphous nanoparticles as a means to control the reduction in particle surface area by irreversible aggregation. STEM is used to illustrate that the aggregates formed by salt or pH flocculation contain primary particle sizes below 1 μm, however their size upon redispersion is 2-10 μm according to static light scattering and corroborated by SEM and BET. The concept of controlled particle growth resulting from irreversible aggregation by specific selection of stabilizers and type of flocculation is not taught by Chen et al.

The preservation of the amorphous morphology throughout the salt and pH flocculation process is not obvious, since typical filtration of nanoparticles takes a period of time sufficient for crystallization. Crystallization of amorphous particles is accelerated by the presence of water and other solvents. However, salt and pH flocculation produce nanoparticle aggregates which separate from the bulk aqueous phase by creaming or settling into a second layer. This separated bulk phase aids in the isolation of particles from water and solvent, which minimizes the time the particles are susceptible to crystallization. Additionally, the flocculation and filtration is conducted at temperatures well below the glass transition temperature of the poorly water-soluble compound (i.e. itraconazole, 58° C.) to minimize mobility of the drug molecules and thus preserve the amorphous morphology even during the additional washing step. In addition, the ability to use rapid filtration and drying eliminates the need to lyophilize the aqueous dispersion to produce a dry powder. Avoiding lyophilization increases the variety of solvents and soluble salts that can be used to initially produce the metastable amorphous nanoparticles. The preservation of amorphous morphology throughout the filtration and additional washing steps was not obvious or anticipated in the previous work by Chen, since that work only discussed crystalline particles.

In some embodiments, the current invention also claims that controlling the growth of particle size, to reduce the surface area of the polymerically stabilized nanoparticles, helps improve and maintain the level of supersaturation attained upon dissolution. Rapidly dissolving amorphous nanoparticles have the potential to raise supersaturation values markedly, relative to more conventional low surface area (<1 m²/g) solid dispersions, by avoiding solvent-mediated crystallization of the undissolved solid. An unanticipated and non-obvious result is that the nanoparticle aggregates created by salt and pH flocculation dissolve as rapidly as individual nanoparticles of the same composition. Additionally, the nanoparticle aggregates produced by salt or pH flocculation are particularly effective for maintaining high supersaturations for several hours, compared to individual nanoparticles, by decreasing the number of heterogeneous sites for nucleation and growth of particles out of the solution. Therefore, the irreversible aggregation of nanoparticles improves the ability of the amorphous drug to supersaturate and maintain levels of supersaturation in aqueous media. Previous work by Chen showed crystalline particles that will not supersaturate aqueous media. However, theoretically if Chen's particles were amorphous, they would still redisperse to their original size and therefore no change in the supersaturation curve would be observed relative to the original nanoparticle dispersion. Thus, Chen et al. do not teach the advancement in dissolution of amorphous nanoparticles.

The polymeric stabilizers may include both non-ionic and pH dependent release polymers flocculated by desolvating the stabilizing moieties by either the addition of a divalent salt or shifting the pH. The use of pH flocculation offers a new type of controlled release of enterically coated amorphous nanoparticle aggregates, where the release of supersaturation may be tuned by control of the aggregate surface area and the choice of enteric polymer. The use of pH dependent release polymers was not discussed in the previous work by Chen et al. and is a second reduction to practice of the current invention.

Overall, the present invention produces a novel way to improve the ability of poorly water-soluble active agent particles to supersaturate aqueous media. The controlled and irreversible aggregation that lead to growth of the nanoparticles, the ability to maintain amorphous morphology even through the additional washing step, the increase in overall ability to supersaturate aqueous media for prolonged periods of time, and a second reduction to practice with flocculation by changing the pH were all unanticipated results making this current invention novel and non-obvious.

The present invention provides a method of forming an amorphous drug-loaded particle by forming one or more amorphous drug-loaded nanoparticles, desolvating the one or more amorphous drug-loaded nanoparticles to form one or more flocculated amorphous drug-loaded nanoparticles, filtering and drying the one or more flocculated amorphous drug-loaded nanoparticles to form amorphous drug-loaded particles. The one or more amorphous drug-loaded nanoparticles include one or more active agents stabilized by one or more polymers.

The present invention also provides a flocculated drug-loaded amorphous nanoparticle. The flocculated drug-loaded amorphous nanoparticle includes one or more active agents and one or more polymer stabilizers that have been desolvated to form one or more flocculated amorphous drug-loaded nanoparticles that form a supersaturated solution of one or more drug-loaded amorphous nanoparticles when resuspended.

The skilled artisan will recognize that the one or more amorphous drug-loaded particles may be formed by precipitation, wet milling, emulsion templating, freezing processes, emulsion processes, spray drying or a combination.

The skilled artisan will recognize that the one or more active agents include itraconazole, Naproxen, capsaicin, cyclosporins, paclitaxel, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiacinotropic agents, contrast media, corticosterioids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasidilators, xanthines, albuterol sulfate, terbutaline sulfate, diphenhydramine hydrochloride, chlorpheniramine maleate, loratidine hydrochloride, fexofenadine hydrochloride, phenylbutazone, nifedipine, carbamazepine, naproxen, cyclosporin, betamethosone, danazol, dexamethasone, prednisone, hydrocortisone, 17 beta-estradiol, ketoconazole, mefenamic acid, beclomethasone, alprazolam, midazolam, miconazole, ibuprofen, ketoprofen, prednisolone, methylprednisone, phenyloin, testosterone, flunisolide, diflunisal, budesonide, fluticasone; proteins, peptides, insulin, glucagon-like peptide, C-Peptide, erythropoietin, calcitonin, human growth hormone, leutenizing hormone, prolactin, adrenocorticotropic hormone, leuprolide, interferon alpha-2b, interferon beta-1a, sargramostim, aldesleukin, interferon alpha-2a, interferon alpha-n3alpha, -proteinase inhibitor; etidronate, nafarelin, chorionic gonadotropin, prostaglandin E2, epoprostenol, acarbose, metformin, or desmopressin, cyclodextrin, antibiotics, antifungal drugs, steroids, anticancer drugs, and the pharmacologically acceptable organic and inorganic salts or metal complexes thereof.

The desolvation may be a result of a variety of processes including increasing salinity with a low molecular weight salt, increasing salinity with a polyelectrolyte, increasing temperature, varying the pH, rinsing with a polymer solution, rinsing with water or a combination thereof.

When the desolvation is caused by the pH being varied the pH may be as low as 1.0 to as high a 7.0 in other cases the pH may be as high as 7.0 to 14.0 depending on the original pH of the solution, the active-agents and/or the polymer. For example, the pH may be 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5 and any incremental variation thereof. In one specific example, the pH may be lowered to a pH of about 2.5.

When the desolvation is caused by increasing salinity with a salt, the skilled artisan will recognize that a monovalent cation, a divalent cation, a trivalent cation a monovalent anion, a divalent anion, trivalent anion or a combination thereof may be used to alter the salinity. Some specific compounds include sodium, potassium, ammonium, calcium, magnesium, sulfate, chloride, fluoride, bromide, iodide, acetate, nitrate, sulfide, phosphate, or a combination thereof. The skilled artisan will recognize that the salts of the Hofineister series may be used to adjust the salinity as this series of salts that have consistent effects on the solubility of proteins and on the stability of their secondary and tertiary structure. Anions appear to have a larger effect than cations, and are usually ordered F—, SO₄²⁻, HPO₄²⁻, Acetate, Cl⁻, NO₃⁻, Br⁻, ClO₃⁻, I⁻, ClO₄⁻, SCN⁻, NH⁴⁺, K⁺, Na⁺, Li⁺, Mg²⁺, Ca²⁺, guanidine and combinations thereof.

The skilled artisan will recognize that the one or more polymers may be ionic or non-ionic polymers. For example, the one or more polymers may be poly(vinylpyrrolidone), PEO, HPMC, PPO, dextran, polysaccharides, polyacrylic acid, polymethacrylic acid, PEO/PPO, copolymers of lactide and glycolide, copolymers containing polyacrylic acid, copolymers containing polymethacrylic acid, copolymers of any of these homopolymers.

The one or more amorphous drug-loaded particles may be about 300 nm in size. However, the skilled artisan will recognize that the one or more amorphous drug-loaded particles may be between 100 nm and 500 nm in size or even less than 100 and greater than 500, e.g., 50, 100, 150, 200, 250, 300, 350, 400, 450, 500, 550, 600, 650, 700, 750 nm or more. Furthermore, the one or more amorphous drug-loaded particles can have a particle diameter that is increased by a factor of between 1.1 and 50 times the diameter of an unflocculated amorphous drug-loaded nanoparticle. More specifically, the one or more amorphous drug-loaded particles have a particle diameter that is increased by a factor of 1.1, 1.2, 1.3, 1.4, 1.5, 2, 2.5, 3, 4, 5, 10, 15, 20, 25, or 50 times the diameter of an unflocculated amorphous drug-loaded nanoparticle.

The skilled artisan will recognize that the one or more amorphous drug-loaded particles may be resuspending to form a supersaturated solution, wherein the resuspended one or more amorphous drug-loaded particles are about the same size as the original one or more amorphous drug-loaded particles, larger than the original one or more amorphous drug-loaded particles, smaller than the original one or more amorphous drug-loaded particles, or a mixture thereof. Generally, the one or more amorphous drug-loaded particles form a supersaturated solution more soluble than a comparable crystalline nanoparticle and the supersaturated solution may be 5 to 20 times more soluble than a comparable crystalline particle. In some instances, the one or more amorphous drug-loaded particles form a supersaturated solution 60 to 90 times more soluble than a comparable crystalline particle, wherein the supersaturated solution has a pH between 1.0-1.4. The one or more amorphous drug-loaded particles are dried to an amorphous drug-loaded cake comprising a drug loading of about 10%, 20%, 30%, 40%, 50%, 60%, 70%, 80%, 90%, and 95%. More specifically, the one or more amorphous drug-loaded particles are dried to an amorphous drug-loaded cake comprising a drug loading of between about 50% and 85% or even greater than 85% or 90%.

The present invention also provides a flocculated drug-loaded amorphous nanoparticle. The flocculated drug-loaded amorphous nanoparticle includes one or more active agents and one or more polymer stabilizers that have been desolvated to form one or more flocculated amorphous drug-loaded nanoparticles that form a supersaturated solution of one or more drug-loaded amorphous nanoparticles when resuspended.

The skilled artisan will recognize that the one or more active agents and one or more polymer stabilizers include one or more amorphous drug-loaded particles formed by precipitation, wet milling, emulsion templating, freezing processes, emulsion processes, spray drying or a combination.

The skilled artisan will recognize that the one or more active agents and one or more polymer stabilizers have been desolvated by an increases in the salinity with a low molecular weight salt, an increases in the salinity with a polyelectrolyte, an increases in the temperature, a variation of the pH, the addition of a polymer solution, the addition of water or a combination thereof.

For poorly water-soluble drugs, formulation into an amorphous form can improve the oral bioavailability by increasing the apparent solubility under physiologically relevant conditions. Higher levels of supersaturation of the drug in the gastrointestinal tract, particularly in the upper intestine, may lead to faster permeation rates through biomembranes and thus, enhance absorption. Solubility of amorphous drugs has been predicted to be as high as 100 to 1600-times larger than the crystalline form on the basis of free energy calculations along with experimental configurational heat capacities. However, nucleation and growth of particles from the supersaturated solution may be detrimental to absorption. Additionally, metastable amorphous and other high energy polymorphs may undergo transitions to lower energy crystalline states in the solid dosage form and/or during dissolution. Consequently supersaturation levels are rarely above 10. Efforts are ongoing to generate and sustain higher supersaturation levels with novel concepts in particle engineering.

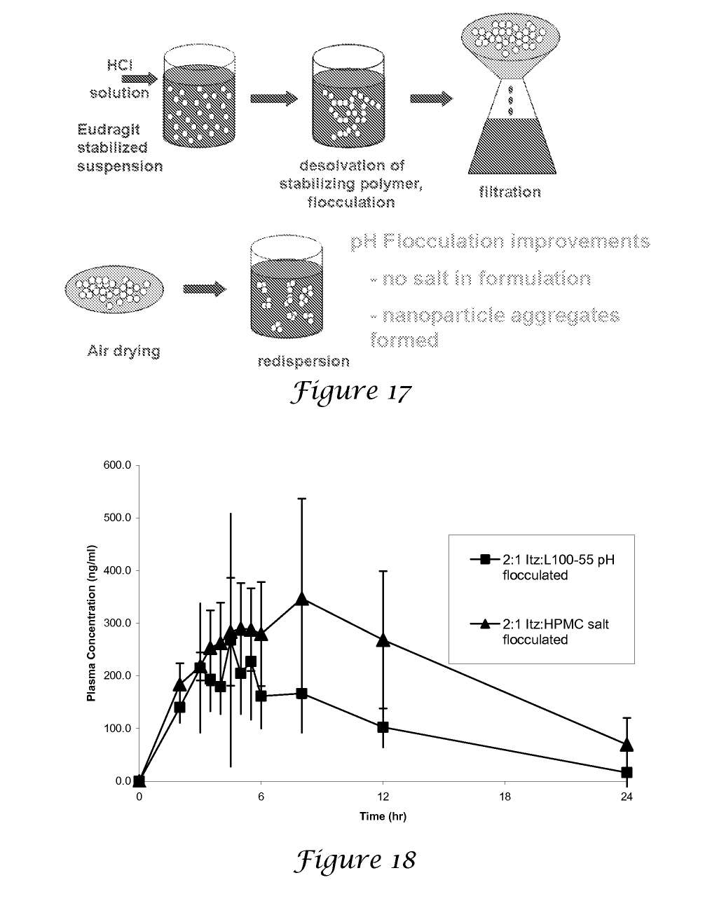

FIG. 17 is a schematic that represents an overview of the process and the table gives a few of the more specific details with regards to potency and salt content of the formulations formed by using the pH flocculation embodiment of this patent. The pH flocculation method includes:

TABLE 12

| | Experimental | Expected | Average | Equivalent | |
|---|---|---|---|---|---|
| | % Itz in | % Itz in | Osmolality | weight of NaCl in | % salt in |
| | sample | sample | (mOsmol/kg) | sample (g) | sample |
| 4:1 Itz:L100-55 | 75.62% | 80.00% | 2 | 0.000011688 | 0.12% |
| pH flocculated | | | | | |
| 2:1 Itz:L100-55 | 66.61% | 66.67% | 0.5 | 0.000002922 | 0.03% |
| pH flocculated | | | | | |

No salt in the final formula. This is advantageous for all types of delivery including oral and parentral because it gives the formulator complete control of the osmolality.

The use of pH buffering salts in the aqueous phase: This removes the need to use additional organic solvents to dissolve polymers that are not easily solubilized in pure water, for example EUDRAGITS®.

Again, as with example 3, the flocculated particles form 1-10 um aggregates upon redispersion. As shown in example 5, these micron sized particles actually give higher overall AUC under 4 hour dissolution due to a decrease in excess surface area that causes nucleation and growth out of the supersaturated media.

Crystallization inhibitors, such as poly(vinylpyrolidone) or hydroxypropylmethylcellulose (HPMC), have been used extensively to form amorphous solid dispersions by solvent evaporation or hot melt extrusion. At loadings (drug wt./tot. wt.) of 50% or less, the drug may be dispersed molecularly in a polymer matrix to prevent the formation of crystalline drug domains. In most cases, evaporation or extrusion is relatively slow and growth leads to particle domains on the order of 100 μm. In contrast, particle engineering by rapid precipitation upon mixing organic solutions into an antisolvent has been utilized to quench poorly water soluble drugs in an amorphous state with surface areas up to 51 m²/g and drug loadings up to 94%. During precipitation, relatively small amounts of polymeric surfactants orient preferentially to the water/drug particle interface to stabilize particles with surface areas on the order of 10-50 m²/g. The rapid dissolution rates of the stabilized high surface area particles limit the time for the undissolved solid phase to crystallize in the presence of the dissolution media resulting in high supersaturations in pH 1.2 media. Supersaturation levels in pH 1.2 media reached 90 within 20 minutes, and decay of supersaturation was inhibited by arresting the growth of embryos with HPMC. Similar growth inhibition was observed in an amorphous 1:1 tacrolimus/HPMC solid dispersion at pH 1.2, where supersaturations of 25 were stable for 24 hours. In contrast, for a low surface area amorphous solid dispersion of a poorly water soluble drug, GWX (proprietary structure), with 60% hydroxypropyl methylcellulose phthalate (HPMCP), crystallization of the solid phase was detected within 1 minutes during the slow dissolution that took place over 60 minutes. Thus, in pH 6.8 media the supersaturation reached only about 3, even with ample amounts of non-ionic stabilizer present.

Because the chemical properties in the stomach are variable and hard to predict, for example pH, quantity of food and residence time, targeting release of drug in the intestine offers the potential of greater control of absorption. For example, for fasted subjects, the residence time in the stomach was 30±26 minutes versus only 2.7±0.8 hours in the small intestine. Also, depending on the fed or fasted state, the residence time in the stomach can range from 30 minutes to 3 hours. The pH can vary from 1.4-2.1 under fasted conditions to 4.3-5.4 in the fed state. Thus, it would be desirable to target amorphous drugs to the upper intestine to form supersaturated solutions, with greater control over the chemical environment, to increase absorption.

Sustained release systems have been formulated for drug delivery throughout the entire GI tract to increase the therapeutic window of crystalline and amorphous poorly water-soluble drugs. Enteric or sustained drug release can be achieved with pH sensitive polymers such as methacrylic acid/methylmethacrylate copolymers, for example EUDRAGITS® or HPMCP or with pH sensitive hydrogels. However, in vitro supersaturation curves at pH values above about 6 are rarely reported, and relatively little is known about how to enhance and sustain supersaturation at these conditions. Furthermore, most previous studies of supersaturation only considered low surface area (<1 m²/g) morphologies, and not particles smaller than about 5 μm.

Recently, we reported that ITZ nanoparticles (300 nm) coated with a nonionic polymeric stabilizer may be recovered from an aqueous dispersion by flocculation with a divalent salt. The micron-sized flocs could then be filtered (1-3 μm pore size) and dried to obtain a powder. The dried powders redispersed in water to their original particle size. This method provides rapid recovery of nanoparticles with minimal residual water to evaporate, and increased drug loading since significant free stabilizer is removed during filtration. Although high supersaturation levels were reported in pH 6.8 media for these particles, the supersaturation mechanism was not investigated in detail.

The present invention provides particles ranging in size from 200 nm to 45 μm to generate high supersaturation levels rapidly in pH 6.8 media and to sustain relatively high levels over 4 hours. To probe supersaturation mechanisms, the behavior is compared for particles with high (>10 m²/g, nanoparticles) and medium (2-5 m²/g, microparticles) surface areas, relative to more commonly studied particles with low (<2 m²/g,) surface area. High surface area particles facilitate rapid dissolution rates of poorly water soluble crystalline and amorphous drugs. However, high surface areas are less important for amorphous relative to crystalline drugs given the greater thermodynamic driving force for dissolution. Undissolved high surface area particles may cause depletion in the level of supersaturation by accelerating heterogeneous nucleation, as well as growth by condensation and coagulation. Thus, medium surface areas may balance two competing effects: (1) sufficient dissolution rate to avoid solvent-mediated crystallization of the undissolved solid phase and (2) minimization of heterogeneous sites for nucleation and growth of particles from the supersaturated solution. Various techniques are presented for the formation of medium surface area amorphous particles, while minimizing the challenging problem of crystallization during particle growth. The particles produced by salt flocculation were particularly effective for maintaining high supersaturations for several hours. The dissolution rates are predicted with reasonable accuracy with a simple mass transfer model developed for ITZ. The effect of undissolved particles on nucleation and growth from supersaturated solution is compared for a wide range in particle surface areas. In a control experiment, ITZ particles are added incrementally to minimize surface area available for growth. To investigate the role of stabilizer charge, a non-ionic stabilizer, HPMC, is compared with an ionic stabilizer containing polymethacrylic acid functionality, EUDRAGITS® L100. Whereas most studies were performed exclusively at pH 6.8, in one case the pH started at 1.2 and it was shifted to pH 6.8 to mimic the transition from the stomach to upper intestine, in particular, as the solubility of ITZ (without surfactant) decreases by 4 orders of magnitude.

B.P. grade itraconazole (ITZ) was purchased from Hawkins, Inc. (Minneapolis, Minn.). HPMC E5 (viscosity of 5 cP at 2% aqueous 25° C. solution) grade was a gift from The Dow Chemical Corporation. Methacrylic acid-methylmethacrylate copolymer (1:1 ratio), EUDRAGIT® L100 (EL100) and methacrylic acid-ethyl acrylate copolymer (1:1 ratio), EUDRAGIT® L100-55 (EL10055) were donated by Degussa Röhm America LLC (Piscataway, N.J.). Drug and polymer chemical structures are given in FIG. 2. Stabilized p.a. grade 1,3-dioxolane was obtained from Acros Organics (Morris Plains, N.J.). HPLC grade acetonitrile (ACN), A.C.S. grade hydrochloric acid (HCl), diethanolamine (DEA), sodium dodecyl sulfate (SDS), sodium sulfate anhydrous (Na₂SO₄), and A.C.S. certified tribasic sodium phosphate (Na₃PO₄) were used as received from Fisher Chemicals (Fairlawn, N.J.).

The method of antisolvent precipitation was used to produce nanoparticle suspensions of ITZ. For HPMC-stabilized particles, deionized water (50 g) containing an appropriate quantity of HPMC was used as the anti-solvent phase into which 15 g of 1,3-dioxolane containing 3.3% (wt) ITZ was injected using a 19G syringe and a flow rate of about 300 mL/min. to form a fine precipitate. The organic phase was separated from the aqueous suspension via vacuum distillation. The aqueous suspension was then added dropwise to liquid nitrogen and lyophilized to form a powder using a Virtis Advantage Tray Lyophilizer (Virtis Company, Gardiner, N.Y.) with 24 hours of primary drying at −35° C. followed by 36 hours of secondary drying at 25° C. ITZ/HPMC particles were also salt flocculated and rapidly filtered, as described by a previous study. Briefly, 120 mL of 1.5M Na₂SO₄ was added to 50 mL of aqueous suspension to form loose flocculates, which could be rapidly filtered in about 10 minutes. The filter cake was dried at about 25° C. and ambient pressure for at least 12 hours. For EL100-stabilized particles, 3.1 g of 4% EL100 in methanol was added to 15 g of 3.3% ITZ solution in 1,3-dioxolane to achieve a 4:1 ratio of ITZ to EL100. The ITZ/EL100 organic solution was then injected into 100 mL of 10⁻⁴N HCl (pH 3.3) to form a co-precipitate. Alternatively, 6.2 g of 2% EL100 in methanol was added dropwise to 100 mL of pure water to form a clear solution. Into the aqueous EL100 solution, 15 g of 3.33% ITZ solution in 1,3-dioxolane was injected to form a fine precipitate. For the 1:1 ITZ/EL10055 formulation, 10 g of 4.8% EL10055 in methanol was added dropwise to 15 g of 3.33% ITZ in 1,3-dioxolane. The resulting clear organic solution was rapidly injected into 100 mL pure deionized water using a 19 G syringe at a flow rate of about 300 mL/min. to form a fine particle dispersion. After organic solvent removal the suspensions were freeze dried as described above.

Approximately 2 grams of ITZ was added to 20 mL of dichloromethane and agitated until completely dissolved. The ITZ solution was placed in a mortar and 1 gram of HPMC was slowly added while gently stirring with a pestle without any precipitation. The solution was stirred gently until approximately 90% of the dichloromethane volume was evaporated, leaving a clear viscous gel. The remaining dichloromethane was removed by heating to 50° C. at a reduced pressure of about 500 mtorr for 2 hours. The resulting drug/polymer film was removed from the mortar and pestle with a straight razor blade and ground to a fine powder for 30 minutes using a ceramic ball mill (1 cm bead size). The final powder was collected after filtration through a size 16 mesh sieve (<1190 μm pore size).

To determine the solubility of crystalline ITZ at 37.2° C., approximately 1.5 mg of bulk ITZ was placed in a glass vials containing 100 mL of pH 6.8 media with 0.17% SDS wt./vol. The media was made by adding 0.2 M tribasic sodium phosphate to 0.1 N HCl at a volume ratio of 1:3, followed by addition of 0.17% SDS. When necessary, the pH was adjusted to 6.8 by addition of 1 N HCl. Two aliquots were removed from each vial after 18 hours, immediately filtered with a 0.2 μm syringe filter and diluted with ACN to double the volume. Drug concentrations were determined by high performance liquid chromatography as described below with at least an n=3.

Rates of supersaturation were measured in pH 6.8 media (as described above) with 0.17% SDS at 37.2° C. A USP paddle method was adapted to accommodate small sample sizes using a VanKel VK6010 Dissolution Tester with a Vanderkamp VK650A heater/circulator (VanKel, Cary, N.C.). Dissolution media (50 mL) were preheated in small 100 mL capacity dissolution vessels (Varian Inc., Cary, N.C.). Dry powder (about 17.6 mg drug) equivalent to approximately 25-times the equilibrium solubility (C_{eq}=14 μg/mL, from solubility study) of ITZ in pH 6.8 buffer with SDS was added to the media at time zero. In some other cases, a smaller dose was added (10.5 mg ITZ, dose=15; 3.5 mg, dose=5). For pH shift experiments, dry powder (about 17.6 mg drug) was added to 60 mL of 0.1 N HCl and 1.0 mL aliquots were taken at 10, 20, 30, 60, and 120 minutes. After 120 minutes, 20 mL of 0.2 M tribasic sodium phosphate with 0.68% SDS was added to shift the pH to 6.8 with a final concentration of 0.17% SDS. Sample aliquots (1.0 mL) were taken at 10, 20, 30, 60, and 120 minutes after the pH shift. For all dissolution experiments, the aliquots were filtered immediately using a 0.2 μm syringe filter and 0.8 mL of the filtrate was subsequently diluted with 0.8 mL of ACN. In all cases, the filtrate was completely clear upon visual inspection and dynamic light scattering of the filtrate gave a count rate of less than 20K cps (too small for particle size analysis). The drug concentration was quantified by high performance liquid chromatography as described below.

ITZ concentrations were quantified using a Shimadzu LC-600 HPLC (Columbia, Md.). The mobile phase was ACN:water:DEA 70:30:0.05 and the flow rate was 1 mL/min. For a detection wavelength of 263 nm, the ITZ peak elution time was 5.4 minutes. The standard curve linearity was verified from 1 to 500 μg/mL with a r² value of at least 0.999.

Dry powder samples were placed on adhesive carbon tape and gold-palladium sputter coated for 45 seconds. Micrographs were taken using a Hitachi S-4500 field emission scanning electron microscope with an accelerating voltage of 15 kV.

Drug crystallinity was detected by a 2920 modulated DSC (TA Instruments, New Castle, Del.) with a refrigerated cooling system. Samples were placed in hermetically sealed aluminum pans and purged with nitrogen at a flow rate of 150 mL/min. The amplitude used was 1° C., the period 1 minute, and the underlying heating rate 5° C./minute.

Powder specific surface areas of drug powder were measured using a Quantichrome Instruments Nova 2000 series surface area analyzer (Boynton Beach, Fla.) using nitrogen as the adsorbate gas. Six points were taken over a range of relative pressures from 0.05 to 0.35. In all cases, correlation coefficients were greater than 0.99, indicating good linear fit with the Brunauer-Emmett-Teller (BET) equation.

A range of particle sizes was produced by AP and solvent evaporation. The flash frozen and lyophilized AP 4:1 and 2:1 ITZ/HPMC dispersions were composed of primary particles that were approximately 200-500 nm in diameter, according to SEM in FIG. 3A, B, and with high surface area (13-17 m²/g from BET). Similar results were reported previously for the same system. According to static light scattering, the primary particles of the original 4:1 ITZ/HPMC AP dispersion formed about 3 μm aggregates. For the flash-frozen lyophilized particles, static light scattering measurements also indicated that the 500 nm primary particles formed aggregates of 2-5 μm upon redispersion in water.

The AP nanoparticle aggregates were also recovered by flocculation with salt and filtration. The salt desolvates the polymeric stabilizers, resulting in strong attractive forces between particles and rapid flocculation to form microparticles on the order of 50 μm. These larger microparticles may be filtered easily. After drying, the salt flocculated particles were redispersed in water to form 10 μm aggregates as characterized by static light scattering. In this case, the primary domains of the salt flocculated aggregates were 2-3 μm in diameter, according to SEM. Therefore, some particle growth occurred during the salt flocculation/filtration process, as the primary domains increased from about 500 nm to about 3 μm. BET surface area measurements were in good agreement with the primary particle size observed by SEM. The values were 13 and 4.4 m²/g for lyophilized and salt flocculated 4:1 ITZ/HPMC, respectively.

In a study, 300 nm primary particles of ITZ stabilized by HPMC and poly(ethylene oxide-b-propylene oxide-b-ethylene oxide) (poloxamer 407) were formed by antisolvent precipitation without any aggregation as observed by static light scattering. The poloxamer 407 provided additional stabilization, as the particles in the current study stabilized by HPMC alone were larger, 500 nm, and were aggregated. The previous dispersions stabilized by HPMC and poloxamer 407 were flocculated by salt and filtered. The primary particle size did not undergo significant growth, according to SEM. When the particles were added to water, static light scattering measurements showed that they redispersed back to their original size of 300 nm indicating the flocculation was reversible. The presence of the second stabilizer, poloxamer 407, provided additional stabilization relative to the ITZ/HPMC particles in the current study. Thus, the salt flocculation process may be used to tune the particle size and surface area by varying the composition of the stabilizers.

Additionally, high (34.6 m²/g) and low (1.24 m²/g) surface area lyophilized 4:1 ITZ/EL100 particles were produced by AP with particle diameters of approximately 200 nm and 15 μm, respectively. In contrast, very large about 45 μm SD particles were produced by solvent evaporation with a surface area <0.1 m²/g.

For the antisolvent precipitation process, the solubility of polymeric stabilizer in the aqueous phase was varied to attempt to manipulate the particle size. To form high surface area nanoparticles, the stabilizer must adsorb to drug particles and be solvated by water to arrest growth. This behavior was achieved for HPMC in water or EL100 in a water/methanol mixture, with primary particles on the order of 200-500 nm. When the aqueous phase was changed to 10⁻⁴ N HCL solution (pH 3.3) without methanol, the acrylic acid groups of EL100 were protonated, rendering the polymer insoluble. With limited diffusion and adsorption of polymer to the drug surfaces, and poor stabilizer solvation, particle growth led to surface areas ranging from 2-4 m²/g. The control of the particle size with polymer solvation has received limited attention in antisolvent precipitation with water soluble stabilizers such as HPMC, polyvinylpyrolidone, and poloxamer 407. Therefore, the ability to tune the solubility of pH dependent polymers such as EL100 is a key advantage for controlling the particle surface area over a wide range.

The morphology of particles produced by AP and solvent evaporation was investigated by DSC, with arrows to indicate crystallization and melting events. The melting temperature of bulk pure ITZ was 168° C. The crystallization of amorphous ITZ was observed upon heating at 115-125° C. For formulations B-D and F-G, the area of the melting peak was approximately equal to that of the crystallization peak. Thus, these formulations were amorphous. In the case of 2:1 ITZ/HPMC AP lyophilized and high surface area 4:1 ITZ/EL100, the crystallization peak was small. However, the drug was significantly amorphous, since the melting peak at 160-168° C. was non existent or very small, compared to pure crystalline ITZ.

Metastable amorphous ITZ may be produced by the rapid AP process, even at drug loadings of 80% (drug wt./tot.wt.). The metastable amorphous state was quenched before drug domains crystallized. This behavior has also been achieved even without any stabilizer present. The nanoparticles remain amorphous throughout the salt flocculation process. The salt flocculation was conducted at about 25° C., well below the glass transition temperature of ITZ (58° C.), to minimize mobility of the drug molecules and mitigate crystallization. The amorphous morphology is preserved as nanoparticles are flocculated to form medium surface area particles at low temperature. In contrast, the significantly higher temperatures in spray drying often produced crystallization of amorphous nanoparticles. The salt flocculated particles will be shown to produce dissolution rates sufficient to generate high sustainable supersaturation levels within minutes.

The dissolution rate was compared for ITZ particles as a function of surface area to investigate the rate of generation of supersaturation, particularly at short times. The complete behavior will be explained more fully in the next section, which will also consider the loss of supersaturation to nucleation and growth from solution. In each study, 17.5 mg of ITZ was added to pH 6.8 media with 0.17% SDS, which corresponds to 25 times the equilibrium solubility of 14 μg/mL. The high and medium surface area particles dissolved rapidly in less than 20 minutes to give supersaturation values ranging from 12 to 17. The peak supersaturation reached 17 for the medium surface area 2:1 ITZ/HPMC salt flocculated AP particles, much higher than typical values of 6 (based on C_{eq} of about 10 μg/mL) for SD particles in pH 6.8 buffer. The total extent of supersaturation was further quantified by calculating the areas under the curve (AUC) using numerical integration. The highest AUC was observed for the medium surface area salt flocculated 4:1 ITZ/HPMC AP particles (1869 minutes). Interestingly, the supersaturation values were lower for the high surface area 4:1 ITZ/HPMC and 4:1 ITZ/EL100 particles (13-36 m²/g). Low surface area 4:1 ITZ/EL100 slowly dissolved to a supersaturation of 6 after 2 hours. Low surface area 4:1 ITZ/HPMC SD particles only reached a supersaturation level of 2.5 in 20 minutes and had the lowest AUC values of all of the samples.

Rapid dissolution shortens the time for crystallization of undissolved particles, offering the potential to increase the maximum supersaturation. In the case of the rapidly dissolving high and medium surface area particles, the maximum supersaturation was much higher than for the slowly dissolving 4:1 ITZ/HPMC SD particles. The design of more rapidly dissolving amorphous particles has the potential to raise supersaturation values markedly relative to more conventional low surface area solid dispersions.

The dissolution rate of a drug particle into a micellar solution is governed by two key steps, (i) micelle uptake of the drug molecules, and (ii) diffusion of the loaded micelle away from the drug particles. According to this model, the initial dissolution rate assuming dilute conditions (bulk concentration is zero) is:

$$\frac{d m}{d t} = {k}_{eff} {AC}_{sat} \qquad ( 3 )$$

where k_{eff} is the overall effective rate constant, and A is the surface area. The value of k_{eff}, which describes uptake from the particle surface into the micelles and diffusion of the drug with the micelles, has been determined to be constant at 0.6 cm/sec for ITZ particle sizes from 200 nm to 2 mm. According to this model, the increase in surface area from 4.4 to 13 m²/g for the salt flocculated and lyophilized 4:1 ITZ/HPMC, respectively, should increase the dissolution rate from 0.4 to 1.5 mg/minutes. In contrast, initial dissolution rates were about the same, 0.84 mg/minutes in each case. However, the large uncertainty may reflect the small number of initial data points. For example, a predicted dissolution rate of 0.4 mg/min, for the medium surface area (4.4 m²/g) microparticles, would produce a supersaturation of 10 within a few minutes. In contrast, observed and predicted rates were identical, at 0.09 mg/min., for the slowly dissolving 4:1 ITZ/HPMC SD particles.

The predicted dissolution rates for high and low surface area 4:1 ITZ/EL100 were 3.0 and 0.1 mg/min., respectively. The observed values of approximately 0.8 and, 0.04 mg/min., respectively, were both somewhat slower than expected, but in the same range. These slower than predicted observed rates may be explained by the presence of the negatively charged EL100 deprotonated methacylic acid groups on the particle surface at pH 6.8. These anions will repel the negatively charged sulfate head groups of SDS micelles to lower the dissolution rate, as observed.

According to equation 3, low surface area 4:1 ITZ/HPMC SD and 4:1 ITZ/EL100 particles should have had similar dissolution rates, however experimentally this was not the case. The difference in their observed dissolution rates can be explained in terms of polymer miscibility. ITZ is miscible with HPMC up to about 50%, thus a large portion of the drug exists in a solid solution within the low surface area polymer matrix. As the ITZ/HPMC SD particles contact the aqueous buffer, HPMC swells with water and the molecularly dispersed drug near the surface will diffuse out rapidly. After this portion of drug dissolves, the supersaturation reaches a maximum as the undissolved drug may crystallize in the slowly dissolving HPMC. Dissolution was slower in the first 20 minutes in the case of ITZ/EL100. Because ITZ miscibility in EUDRAGITS® is only 13% according to mDSC studies, relatively large ITZ domains are phase separated from the polymer phase. Over the first 20 minutes, these large drug domains dissolved more slowly than the molecularly dispersed drug in the ITZ/HPMC. Despite their slower initial dissolution, low surface area EL100-stabilized particles attained a higher level in supersaturation than ITZ/HPMC SD.

Slow dissolution typically allows time for solvent mediated crystallization, as has been observed in previous studies of low surface area SD particles. Such crystallization reduced supersaturation levels. If the SD contains a large amount of stabilizing polymer, as in the case of amorphous 1:1 tacrolimus/HPMC, crystallization of the solid phase may be limited as supersaturation levels were 25 in pH 1.2 media. For higher drug loading, the amount of excipient may become too small to inhibit crystallization. For the low surface area 4:1 particles of this work, the polymer content is 20% and relatively low levels in supersaturation were observed for ITZ/HPMC compared to ITZ/EL100. The better protection against crystallization for EL100 versus HPMC, may indicate stronger binding between the ITZ and EL100 to prevent crystal growth. The negatively charged acrylic acid groups will bind with Lewis acid sites on the ITZ. Furthermore, the more rapid dissolution of EL100 versus HPMC may leader to a greater concentration of dissolved polymer chains to adsorb on the undissolved drug particles to passivate growth of crystalline domains and to provide electrostatic stabilization of particle-particle interactions. Finally, the large amount of undissolved HPMC (relative to fast dissolving EL100) may act as nucleation sites for crystallization of the undissolved amorphous drug.

The dose of particles added to the dissolution media was varied to manipulate the excess surface area of undissolved particles. An increase in excess surface area may accelerate the rate of depletion of the supersaturation by enhancing nucleation and growth rates. For high surface area lyophilized 2:1 ITZ/HPMC, the dose was varied from 25 to 5. The excess surface areas corresponding to doses of 25, 15, and 5 were 0.44, 0.27, and 0.09 m² respectively (corresponding to the minimum supersaturation in each case). The rate of depletion in supersaturation was estimated by taking the initial slope of the depletion phase in supersaturation curves, starting at the maximum in drug concentration. At the lowest dose of 5, the supersaturation of only 4 produced a relatively small driving force for nucleation and growth and thus decay in supersaturation. At a higher dose of 15, the supersaturation reached a much higher level of 13, resulting in a much faster depletion rate of about 0.17 min⁻¹ The higher supersaturation of 13 provided a larger driving force for faster nucleation and growth rates. In addition, the higher excess surface area of undissolved particles also produced faster growth rates by condensation and coagulation. Finally, the highest supersaturation and depletion rates were observed for the largest dose of 25, continuing the trends seen for the increase in dose from 5 to 15.

In a control study, high surface area 2:1 ITZ/HPMC powder was added incrementally, in small doses to avoid building up excess surface area. An initial dose of 5 mg dissolved completely with a linear slope over 1 hr. (results not shown). An additional dose of 2 mg was added after 1 hour and again after 90 minutes for a total dose of 9 mg in 80 mL. All of the added particles dissolved, as indicated by the supersaturation level of 8 (112 μg/mL), which is identical to the total added dose, for an excess surface area of essentially 0. In a similar control experiment, undissolved particles were removed after 10 minutes of dissolution to attempt to minimize decay in the supersaturation. Here 20 mg of 1:1 ITZ/EL10055 particles were added to 50 mL of pH 6.8 media with 0.17% SDS and the entire volume was filtered with a 0.2 μm syringe filter after 10 minutes to remove all the undissolved particles. HPLC analysis verified that a supersaturation of 12.5 was sustained for 30 minutes without any precipitation. In each of these control study, supersaturation levels did not decay when excess particles were absent. Homogeneous nucleation rates appeared to be too slow to deplete the supersaturation levels of 8-12, in the absence of significant excess particle surface area. Therefore, depletion rates in supersaturation are much slower in media where excess surface area from undissolved particles is minimal, as will be elaborated upon further in the following discussion.

At a constant dose of 25, high surface area lyophilized 4:1 and 2:1 ITZ/HPMC rapidly dissolved to a supersaturation of 12-16, followed by depletion to about 3 within 1 hour. Likewise, high surface area 4:1 ITZ/EL100 particles dissolved to a supersaturation of 12 within 10 minutes and precipitated to 5 after 1 hour. For these high surface area particles, the excess surface areas were 0.28, 0.44, and 0.76 m², based on the initial dose. However, the resulting rapid nucleation rates followed by growth from high excess surface areas depleted the supersaturation within the first hour of dissolution. Very similar behavior was observed for the high surface area 300 nm particles produced by salt flocculation of ITZ stabilized by HPMC and poloxamer 407, when the dose was 25. An alternative technique was utilized to attempt to prevent the rapid decay of supersaturation. The original nanoparticle dispersion was added dropwise to prevent buildup of excess particles and stopped when the solution started to become turbid at a dosage of about 60. As a result of the small amount of excess surface area of undissolved particles, growth was inhibited yielding supersaturations of 11 and 7.5 at 20 minutes and 2 hours, respectively. Otherwise, the use of particles with high surface areas was detrimental for long-term maintenance of supersaturation, despite the benefit of the initial rapid dissolution to produce a high maximum supersaturation.

Low surface area 4:1 ITZ/HPMC SD and 4:1 ITZ/EL100 particles produced a minimal amount of excess surface area for a dose of 25. In the case of these slowly dissolving low surface area particles, the relatively low maximum supersaturation levels led to a low AUC, particularly for the HPMC case. The low excess surface area produced slow rates of growth from solution by condensation and coagulation, even for the relatively high supersaturation of 6 for 4:1 ITZ/EL100. The relatively stable and high supersaturation level of 6 for 2 hours indicates the advantage of electrostatic stabilizers to prevent growth while mitigating crystallization during dissolution. Furthermore, the more rapid dissolution of EL100 versus HPMC may leader to a greater concentration of dissolved polymer chains to adsorb on the undissolved drug particles to passivate growth of crystalline domains and to provide electrostatic stabilization of particle-particle interactions. Finally, the large amount of undissolved HPMC (relative to fast dissolving EL100) may act as nucleation sites for crystallization of the undissolved amorphous drug.

Medium surface area particles produced the highest AUC values in this work, particularly the salt flocculated particles. Despite the increase in the primary particle size of amorphous ITZ from about 500 nm to about 3 μm according to SEMs, the dissolution was still sufficiently rapid to generate high supersaturation levels as discussed above. In addition the excess surface area of undissolved particles was sufficiently low to mitigate decay of supersaturation via nucleation and growth by condensation and coagulation. For example, for 4:1 ITZ/HPMC, the supersaturation was still 6 after 2 hours versus only about 3 for the lyophilized sample with a corresponding AUC of 1869 versus 845 minutes. The decay in supersaturation was much slower than for the high surface area lyophilized formulations, despite the identical AP starting material and same composition. The extended level of supersaturation for the 4:1 ITZ/HPMC salt flocculated sample was achieved despite the very small amount of HPMC.

On the basis of the similar peak supersaturations alone, the driving force for nucleation and growth would have been similar for both lyophilized and salt flocculated particles. However, the lower surface area of the salt flocculated particles, by a factor of 0.34 led to slower growth by condensation and coagulation, by factors of 0.34 and 0.039, respectively. Thus, the medium surface area, salt flocculated particles offer an optimal balance of a sufficiently rapid dissolution rate, along with only moderate decay of supersaturation from excess surface area. To our knowledge, this concept has not been reported previously.

Recovery of particles by salt flocculation offers other advantages, in addition to producing high supersaturation levels in pH 6.8 media. The particles may be recovered at 25° C., relative temperatures >90° C. in spray drying. This high temperature crystallized amorphous ITZ nanoparticles in aqueous dispersion, whereas they remained amorphous during salt flocculation. After salt flocculation, the dried particles have been shown to contain less than about 1% residual salt. These particles are more hydrophilic and more efficiently wetted by aqueous media than lyophilized powders, as is evident from smaller contact angles. Finally the salt flocculation process produces higher yields and reduces the energy requirements relative to spray drying.

To mimic the release of drug in the stomach followed by the transition to the upper intestine, selected formulations were dissolved in pH 1.2 media for 2 hours, and the pH was shifted to 6.8. The goal was to investigate how the quantity of dissolved drug at pH 1.2 affects the supersaturation behavior upon shifting to pH 6.8. The equilibrium solubilities were 4.4 and 14 μg/mL in the acidic and neutral media, respectively. The latter would have been only about 1 ng/mL, in buffer without the addition of 0.17% SDS. Lyophilized 4:1 ITZ/HPMC and an enteric-type release 1:1 ITZ/EL10055 formulation were compared to the commercial solid ITZ product, SPORANOX®, which is a 20% (wt.) ITZ formulation including HPMC as a stabilizer. A constant drug dose of 350 μg/mL (based on volume of media before the pH shift) was added to the pH 1.2 media in each case.

High surface area lyophilized 4:1 ITZ/HPMC dissolved in pH 1.2 media to yield a supersaturation of about 12.5 (based on 14 mg/mL) and precipitated to only 5 even after 2 hours at pH 6.8. When the same lyophilized 4:1 ITZ dose was added directly to pH 6.8 media, the supersaturation decayed more rapidly from 12 to 4 after only 20 minutes. Several factors contribute to the superior stability in supersaturation for the pH shift experiment. For example, approximately half of the mass dissolved at pH 1.2, which reduced the excess surface area available for precipitation from condensation and coagulation upon pH shift. Also, the HPMC had 2 hours to dissolve in the acidic phase. Thus the amorphous drug was not trapped in a swollen slowly dissolving HPMC gel at pH 6.8, where it is likely to crystallize. Also the dissolved HPMC was then available to stabilize embryos, which nucleated upon pH shift to 6.8, and slow down growth. Therefore, the dissolution of particles in pH 1.2 media to moderate supersaturation levels may reduce the depletion rate of supersaturation upon pH shift relative to direct dissolution in pH 6.8 media alone.

For the EL10055 formulation, ITZ dissolution was slowed down at pH 1.2, where the protonated nonionic polymer is insoluble, as shown in FIG. 8. Upon pH shift to 6.8, the particles rapidly dissolved to about 230 μg/mL followed by precipitation to about 40 μg/mL within 2 hours. The supersaturation profile after the maximum was similar to that of high surface area 4:1 ITZ/EL100 dissolved in pH 6.8 media alone. In both cases, the high supersaturation created a large driving force for nucleation and growth in the presence of the excess high surface area of undissolved particles.

The dissolution of ITZ in SPORANOX® pellets in pH 1.2 media was rapid and almost complete within 2 hours to produce a supersaturation of 22 (based on C_{eq}=14 μg/mL at pH 6.8). Depletion to 40 μg/mL (supersaturation <3) occurred within 20 minutes after pH shift to pH 6.8. Although the shift in pH caused an increase in drug solubility from 4.4 to 14 μg/mL, the extremely high supersaturation appeared to produce fast nucleation rates and subsequent growth at pH 6.8. As many nuclei were formed and grew, the dissolved HPMC was unable to adsorb to a sufficient level to protect against growth, as was evident by the rapid decay of supersaturation. From the results for the lyophilized 4:1 ITZ/HPMC particles, it is apparent that dissolution to moderate supersaturation levels under acidic conditions eliminates some excess surface area resulting is slower decay of supersaturation at pH 6.8. The maximization of supersaturation levels throughout the gastrointestinal tract may be expected to enhance absorption of poorly water soluble drugs.

Both high and medium surface area amorphous particles, recovered from aqueous dispersions of nanoparticle aggregates formed by antisolvent precipitation, rapidly dissolved in pH 6.8 media to generate supersaturation levels as high as 17 within 10 minutes. Medium surface area (2-5 m²/g) microparticles of amorphous drug were recovered from aqueous dispersions of nanoparticle aggregates by flocculation with salt and filtration, whereas high surface area (13-36 m²/g) particles were obtained by lyophilization. SEM indicated the primary particles grew to about 3 μm during salt flocculation to lower the surface area relative to the identical dispersions dried by lyophilization. Thus, the salt flocculation/filtration process may be used to tune the particle size and surface area. The rapid dissolution, before the undissolved particles crystallized, led to much higher maximum levels in supersaturation for the medium surface area particles relative to more conventional low surface area solid dispersions. The decay in supersaturation was much slower for the medium relative to the high surface area particles, as a consequence of the lower excess surface area of undissolved particles, which act as sites for growth by condensation and coagulation. A similar result was achieved by initially dissolving part of the drug at pH 1.2 to reduce the excess surface area, and then shifting the pH to 6.8. This pH shift mimics the transition from the stomach to the intestines. Furthermore, the decay in supersaturation essentially vanished when the excess surface area of undissolved particles was negligible. Amorphous medium surface area microparticles which dissolve rapidly to produce high supersaturation values with slow decay offer the potential for improved gastrointestinal absorption and thus enhanced bioavailability.

Aqueous suspensions of crystalline naproxen nanoparticles, formed by antisolvent precipitation, were flocculated with sodium sulfate, filtered and dried to form redispersible powders for oral delivery. The particles were stabilized with polyvinylpyrrolidone (PVP K-15) and/or poly(ethylene oxide-b-propylene oxide-b-ethylene oxide) (poloxamer 407). The yield of the drug in the powder was typically 92 to 99%, and the drug potency was reproducible to within 1 to 2%. The filtration process increased the drug potency by up to 61% relative to the initial value, as unbound surfactant was removed with the filtrate. Upon redispersion of the dried powder, the average particle size measured by light scattering was comparable to the value in the aqueous suspension prior to flocculation, and consistent with primary particle sizes observed by SEM. For 300 nm particles, extremely rapid dissolution of up to 95% of the drug was dissolved in two minutes. The dissolution rate was linearly correlated with the specific surface area calculated from the average particle diameter after redispersion. The redispersibility of dried powders was examined as a function of the salt concentration used for flocculation and the surfactant composition and concentration. The residual sodium sulfate concentration in the dried powders was far below the toxic limit. Flocculation followed by filtration and drying is an efficient and highly reproducible process for the rapid recovery of drug nanoparticles to produce wettable powders with high drug potency and high dissolution rates.

Flocculation by shifting pH of a pH-sensitive polymer coated nanoparticle. An organic solution of 3.33% itraconazole in 1,3-dioxolane was added using a 19G needle and syringe to a buffered solution containing various amounts of dissolved EUDRAGIT® L100-55. Immediately after precipitation, sufficient quantities of 0.1 N HCl was added to lower the pH to approximately 2.5, which caused immediate flocculation of the nanoparticles. The flocculated nanoparticles were then vacuum filtered with fine porosity P2 type Fisherbrand filters (pore size approximately 5 μm). The filtrate was clear and the filter cake was dried at ambient conditions overnight. The filter cake was then removed from the filter and stored in ambient conditions. A sample of the powder was dissolved in 20 mL of a 70:30 mixture of acetonitrile and water and 10 mL of methanol. This sample was then diluted in half with acetonitrile and tested for itraconazole concentration by HPLC. A separate sample of 0.01 g of the powder was exposed and stirred in 0.2 mL of DI water and left for 3 days. The sample was then filtered using a 0.2 μm filter and placed in analysis tubes for the μOsmette. Osmolality measurements were used to quantify the salt concentration in the sample by assuming that any salt in the formulation was sodium chloride. Results for the drug loading, osmolality and % salt in the sample are found in table 13 below.

| | Experimental | Average | |
|---|---|---|---|
| | % Itz | Osmolality | % salt in |
| | in sample | (mOsmol/kg) | sample |
| 4:1 Itz:L100-55 pH | 75.62% | 2 | 0.12% |
| flocculated | | | |
| 2:1 Itz:L100-55 pH | 66.61% | 0.5 | 0.03% |
| flocculated | | | |

Animal Study using Sprague-Dawley Rats: Jugular vein pre-catheterized male Sprague Dawley rats weighing approximately 300 g each were subdivided into groups of 6 to be dosed via oral gavage with 1 mL suspension containing 15 mg itraconazole/kg rat of a selected itraconazole powders produced as described in Examples 3 and Table 13. Blood samples were withdrawn from the animals at designated timepoints and analyzed for itraconazole plasma concentration using HPLC and a ketoconazole internal standard. The results for the itraconazole plasma concentration versus time is shown in FIG. 18 and the statistical analysis of the results for the peak concentration (Cmax), the time of the peak concentration (Tmax) and the area under the curve (AUC) is shown in table 14. See Table 14 below:

| | Cmax | | AUC |
|---|---|---|---|
| Sample | (ng/mL) | Tmax (hr) | (ng * hr/mL) |
| 2:1 Itz:HPMC | 388.9 +/− 169 | 8.25 +/− 3.3 | 5084 +/− 1970 |
| salt flocculated | | | |
| 2:1 Itz:Eudragit | 350.4 +/− 226.4 | 4.3 +/− 1.4 | 2526 +/− 714 |
| L100-55 pH | | | |
| flocculated | | | |

FIG. 18 is a plot of the average plasma concentration of ITZ in rats over 24 after administration of a dispersion of two nanoparticle aggregate formulations and SPORANOX® capsules.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of forming one or more amorphous drug-loaded particle comprising the steps of:
forming one or more amorphous drug-loaded nanoparticles comprising one or more active agents stabilized by one or more polymers;
rapidly desolvating the one or more amorphous drug-loaded nanoparticles to form one or more flocculated amorphous drug-loaded nanoparticles;
filtering the one or more flocculated amorphous drug-loaded nanoparticles; and
drying the one or more flocculated amorphous drug-loaded nanoparticles to form the one or more amorphous drug-loaded particles.

2. The method of claim 1, wherein the one or more amorphous drug-loaded particles are formed by precipitation, wet milling, emulsion templating, freezing processes, emulsion processes, spray drying or a combination.

3. The method of claim 1, wherein the one or more active agents comprise itraconazole, danazol, paclitaxel, cyclosporin, naproxen, capsaicin, albuterol sulfate, terbutaline sulfate, diphenhydramine hydrochloride, chlorpheniramine maleate, loratidine hydrochloride, fexofenadine hydrochloride, phenylbutazone, nifedipine, carbamazepine, naproxen, cyclosporin, betamethosone, danazol, dexamethasone, prednisone, hydrocortisone, 17 beta-estradiol, ketoconazole, mefenamic acid, beclomethasone, alprazolam, midazolam, miconazole, ibuprofen, ketoprofen, prednisolone, methylprednisone, phenyloin, testosterone, flunisolide, diflunisal, budesonide, fluticasone; proteins, peptides, insulin, glucagon-like peptide, C-Peptide, erythropoietin, calcitonin, human growth hormone, leutenizing hormone, prolactin, adrenocorticotropic hormone, leuprolide, interferon alpha-2b, interferon beta-1a, sargramostim, aldesleukin, interferon alpha-2a, interferon alpha-n3alpha, -proteinase inhibitor; etidronate, nafarelin, chorionic gonadotropin, prostaglandin E2, epoprostenol, acarbose, metformin, or desmopressin, cyclodextrin, antibiotics, antifungal drugs, steroids, anticancer drugs, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiacinotropic agents, contrast media, corticosterioids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasidilators and xanthines and the pharmacologically acceptable organic and inorganic salts or metal complexes thereof.

4. The method of claim 1, wherein the desolvation is caused by increasing salinity with a low molecular weight salt, increasing salinity with a polyelectrolyte, increasing temperature, or varying the pH, rinsing with a polymer solution, rinsing with water or a combination thereof.

5. The method of claim 1, wherein the pH is lowered to about 2.5 to desolvate a pH sensitive polymer.

6. The method of claim 1, wherein the desolvation is caused by increasing salinity with a salt comprising a monovalent, divalent or trivalent cations, a monovalent, divalent, or trivalent anions or a combination thereof.

7. The method of claim 1, wherein the desolvation is caused by increasing salinity with a salt comprising sodium, potassium, ammonium, calcium, aluminum, iron, magnesium, sulfate, chloride, fluoride, bromide, iodide, acetate, nitrate, sulfide, phosphate, carbonate, aluminate, silicate, oxide, hydroxide or a combination thereof.

8. The method of claim 1, wherein the one or more polymers comprise non-ionic polymers.

9. The method of claim 1, wherein the one or more polymers comprises poly(vinylpyrrolidone), PEO, HPMC, PPO, dextran, polysaccharides, polyacrylic acid, polymethacrylic acid, polyacrylamide, PEO/PPO, albumin, chitosan, peptides, papain, collagens, copolymers of lactide and glycolide, copolymers containing polyacrylic acid, copolymers containing polymethacrylic acid, copolymers of any of these homopolymers, copolymers of these homopolymers with the addition of other homopolymers and copolymers.

10. The method of claim 1, wherein the one or more amorphous drug-loaded particles are about 300 nm in size.

11. The method of claim 1, wherein the one or more amorphous drug-loaded particles are between 100 nm and 500 nm in size.

12. The method of claim 1, wherein the one or more amorphous drug-loaded particles have a particle diameter that is increased by a factor of between 1.1 and 50 times the diameter of an unflocculated amorphous drug-loaded nanoparticle.

13. The method of claim 1, wherein the one or more amorphous drug-loaded particles have a particle diameter that is increased by a factor of 1.1, 1.2, 1.3, 1.4, 1.5, 2, 2.5, 3, 4, 5, 10, 15, 20, 25, or 50 times the diameter of an unflocculated amorphous drug-loaded nanoparticle.

14. The method of claim 1, further comprising the step of resuspending the one or more amorphous drug-loaded particles to form a supersaturated solution, wherein the resuspended one or more amorphous drug-loaded particles are about the same size as the original one or more amorphous drug-loaded particles.

15. The method of claim 1, further comprising the step of resuspending the one or more amorphous drug-loaded particles to form a supersaturated solution, wherein the resuspended one or more amorphous drug-loaded particles are larger than the original one or more amorphous drug-loaded particles.

16. The method of claim 1, wherein the one or more amorphous drug-loaded particles form a supersaturated solution more soluble than a comparable crystalline nanoparticle.

17. The method of claim 1, wherein the one or more amorphous drug-loaded particles form a supersaturated solution 5 to 20 times more soluble than a comparable crystalline particle in a pH 6.8 aqueous media with 0.17% SDS added.

18. The method of claim 1, wherein the one or more amorphous drug-loaded particles form a supersaturated solution 60 to 90 times more soluble than a comparable crystalline particle, wherein the supersaturated solution has a pH between 1.0-1.4.

19. The method of claim 1, wherein the one or more amorphous drug-loaded particles are dried to an amorphous drug-loaded cake comprising a drug loading of about 10%, 20%, 30%, 40%, 50%, 60%, 70%, 80%, 90%, and 95%.

20. The method of claim 1, wherein the one or more amorphous drug-loaded particles are dried to an amorphous drug-loaded cake comprising a drug loading of between about 50% and 85%.

21. The method of claim 1, wherein the one or more amorphous drug-loaded particles are dried to an amorphous drug-loaded cake comprising a drug loading of greater than 85%.

22. The method of claim 1, wherein the one or more amorphous drug-loaded particles are dried to an amorphous drug-loaded cake comprising a drug loading of greater than 90%.

23. An amorphous salt-flocculated nanoparticle formed by the method of claim 1.

24. A flocculated drug-loaded amorphous nanoparticle comprising:
one or more active agents and one or more polymer stabilizers that have been desolvated to form one or more flocculated amorphous drug-loaded nanoparticles that form a supersaturated solution of one or more drug-loaded amorphous nanoparticles when resuspended.

25. The nanoparticle of claim 24, wherein the one or more active agents and one or more polymer stabilizers comprise one or more amorphous drug-loaded particles formed by precipitation, wet milling, emulsion templating, freezing processes, emulsion processes, spray drying or a combination.

26. The nanoparticle of claim 24, wherein the one or more active agents comprise itraconazole, Naproxen, capsaicin albuterol sulfate, terbutaline sulfate, diphenhydramine hydrochloride, chlorpheniramine maleate, loratidine hydrochloride, fexofenadine hydrochloride, phenylbutazone, nifedipine, carbamazepine, naproxen, cyclosporin, betamethosone, danazol, dexamethasone, prednisone, hydrocortisone, 17 beta-estradiol, ketoconazole, mefenamic acid, beclomethasone, alprazolam, midazolam, miconazole, ibuprofen, ketoprofen, prednisolone, methylprednisone, phenyloin, testosterone, flunisolide, diflunisal, budesonide, fluticasone; proteins, peptides, insulin, glucagon-like peptide, C-Peptide, erythropoietin, calcitonin, human growth hormone, leutenizing hormone, prolactin, adrenocorticotropic hormone, leuprolide, interferon alpha-2b, interferon beta-1a, sargramostim, aldesleukin, interferon alpha-2a, interferon alpha-n3alpha, -proteinase inhibitor; etidronate, nafarelin, chorionic gonadotropin, prostaglandin E2, epoprostenol, acarbose, metformin, or desmopressin, cyclodextrin, antibiotics, antifungal drugs, steroids, anticancer drugs, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiacinotropic agents, contrast media, corticosterioids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasidilators and xanthines and the pharmacologically acceptable organic and inorganic salts or metal complex thereof.

27. The nanoparticle of claim 24, wherein the one or more active agents and one or more polymer stabilizers have been desolvated by an increases in the salinity with a low molecular weight salt, an increase in the salinity with a polyelectrolyte, an increase in the temperature, a variation of the pH, the addition of a polymer solution, the addition of water or a combination thereof.

28. The nanoparticle of claim 24 wherein the one or more active agents and one or more polymer stabilizers have been desolvated by a pH decrease to about 2.5.

29. The nanoparticle of claim 24, wherein the one or more active agents and one or more polymer stabilizers have been desolvated by increasing salinity with a salt comprising monovalent cations, divalent cations, trivalent anions or a combination thereof.

30. The nanoparticle of claim 24, wherein the one or more active agents and one or more polymer stabilizers have been desolvated by increasing salinity with a salt comprising sodium, potassium, ammonium, calcium, magnesium, sulfate, chloride, fluoride, bromide, iodide, acetate, nitrate, sulfide, phosphate, or a combination thereof.

31. The nanoparticle of claim 24, wherein the one or more polymers stabilizers comprise non-ionic polymers.

32. The nanoparticle of claim 24, wherein the one or more polymers stabilizers comprises poly(vinylpyrrolidone), PEO, HPMC, PPO, dextran, polysaccharides, polyacrylic acid, polymethacrylic acid, PEO/PPO, copolymers of lactide and glycolide, copolymers containing polyacrylic acid, copolymers containing polymethacrylic acid, copolymers of any of these homopolymers.

33. The nanoparticle of claim 24, wherein the one or more amorphous drug-loaded particles are between 100 nm and 500 nm in size.

34. The nanoparticle of claim 24, wherein the one or more amorphous drug-loaded particles have a particle diameter that is increased by a factor of between 1.1 and 50 times the diameter of an unflocculated amorphous drug-loaded nanoparticle.

35. The nanoparticle of claim 24, wherein the one or more amorphous drug-loaded particles are resuspended to form a supersaturated solution.

36. The nanoparticle of claim 24, wherein the one or more amorphous drug-loaded particles form a supersaturated solution more soluble than a comparable crystalline nanoparticle.

37. The nanoparticle of claim 24, wherein the one or more amorphous drug-loaded particles form a supersaturated solution 5 to 20 times more soluble than a comparable crystalline particle.

38. An amorphous salt-flocculated nanoparticle formed by the method of claim 24.

39. A method of increasing the bioavailability of an active agent in a subject comprising the steps of:
administering to a subject an amorphous drug-loaded floc comprising one or more active agents and one or more polymer stabilizers that have been desolvated to form one or more flocculated amorphous drug-loaded nanoparticles that form a supersaturated solution of one or more drug-loaded amorphous nanoparticles when resuspended.

40. A method of increasing the concentration of an active agent in a subject comprising the steps of:
administering to a subject one or more flocculated amorphous drug-loaded particles comprising one or more active agents and one or more polymer stabilizers that have been desolvated to form one or more flocculated amorphous drug-loaded nanoparticles that form a supersaturated solution of one or more drug-loaded amorphous nanoparticles when administered to a subject.

41. An amorphous drug-loaded particle floc formed by the process comprising the steps:
forming one or more amorphous drug-loaded nanoparticles comprising one or more active agents stabilized by one or more polymers;
desolvating the one or more amorphous drug-loaded nanoparticles to form one or more flocculated amorphous drug-loaded nanoparticles;
filtering the one or more flocculated amorphous drug-loaded nanoparticles; and
drying the one or more flocculated amorphous drug-loaded nanoparticles to form amorphous drug-loaded particles, wherein the one or more flocculated amorphous drug-loaded nanoparticles achieve a supersaturated solution when resuspended.

42. A method of forming a redispersible floc comprising the steps of:
forming one or more amorphous drug-loaded nanoparticles comprising one or more active agents stabilized by one or more polymers;
desolvating the one or more amorphous drug-loaded nanoparticles to form one or more flocculated amorphous drug-loaded nanoparticles;
filtering the one or more flocculated amorphous drug-loaded nanoparticles; and
drying the one or more flocculated amorphous drug-loaded nanoparticles to form one or more amorphous drug-loaded particles.
